(19) United States
(12) Patent Application Publication
(10) Pub. No.: US 2024/0058316 A1
(43) Pub. Date: Feb. 22, 2024

(54) PRODRUG OF A PHENOLIC TRPV1 AGONIST FOR THE TREATMENT OF PAIN

(71) Applicant: Concentric Analgesics, Inc., San Francisco, CA (US)

(72) Inventors: Sam L. TEICHMAN, San Francisco, CA (US); Susan KRAMER, San Francisco, CA (US); John F. DONOVAN, San Francisco, CA (US)

(21) Appl. No.: 18/257,275

(22) PCT Filed: Dec. 13, 2021
(86) PCT No.: PCT/US2021/063126

§ 371 Date: Jun. 13, 2023

Related U.S. Application Data
(60) Provisional application No. 63/125,307, filed on Dec. 14, 2020.

(51) Int. Cl. A61K 31/445; A61P 25/04

(52) U.S. Cl. CPC A61K 31/445; A61P 25/04

(57) ABSTRACT

Described herein is a prodrug of a transient receptor potential vanilloid 1 receptor (TRPV1) agonist for the treatment of pain.

### CROSS-REFERENCE

This application claims benefit of U.S. Provisional Patent Application No. 63/125,307, filed on Dec. 14, 2020 which is incorporated herein by reference in its entirety.

### BACKGROUND OF THE INVENTION

More than 80% of patients who undergo surgical procedures experience acute postoperative pain and approximately 75% of those with postoperative pain report the severity as moderate, severe, or extreme (Apfelbaum et al., 2003; Gan et al., 2014). Evidence suggests that less than half of patients who undergo surgery report adequate postoperative pain relief (Apfelbaum et al., 2003). Inadequately controlled pain negatively affects quality of life, function, and functional recovery, the risk of post-surgical complications, and the risk of persistent postsurgical pain (Kehlet et al., 2006). Thus, there exists a need for medicaments with improved efficacy and longer duration of action for the treatment of pain.

### SUMMARY OF THE INVENTION

In one aspect, described herein is a method of treating pain in a subject undergoing surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.05 mg/mL to 0.5 mg/mL. In some embodiments is a method of treating pain in a subject undergoing surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.1 mg/mL to 0.4 mg/mL. In some embodiments is a method of treating pain in a subject undergoing surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.15 mg/mL to 0.35 mg/mL. In some embodiments is a method of treating pain in a subject undergoing surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.2 mg/mL to 0.3 mg/mL. In some embodiments, the pain is from orthopedic surgery. In some embodiments, the pain is from a bunionectomy. In some embodiments, the pain is from a unilateral total knee arthroplasty (TKA). In some embodiments, the pain is from a laparotomy. In some embodiments, the pain is from a laparotomy to repair a ventral hernia.

In another aspect, described herein is a method of treating pain in a subject undergoing a bunionectomy comprising administering to the subject 0.5 mg to 5.0 mg of (E)-2-methoxy-4-((8-methylnon-6-enamido)methyl)phenyl 2-((methylamino)methyl)piperidine-1-carboxylate (Compound 1).

In another aspect, described herein is a method of treating pain in a subject undergoing a bunionectomy comprising administering to the subject 0.5 mg to 5.0 mg of (E)-2-methoxy-4-((8-methylnon-6-enamido)methyl)phenyl 2-((methylamino)methyl)piperidine-1-carboxylate (Compound 1), wherein the pain is reduced by at least 15% compared to placebo. In some embodiments is a method of treating pain in a subject undergoing a bunionectomy comprising administering to the subject 0.5 mg to 5.0 mg of (E)-2-methoxy-4-((8-methylnon-6-enamido)methyl)phenyl 2-((methylamino)methyl)piperidine-1-carboxylate (Compound 1), wherein the pain is reduced by at least 20% compared to placebo. In some embodiments is a method of treating pain in a subject undergoing a bunionectomy comprising administering to the subject 0.5 mg to 5.0 mg of (E)-2-methoxy-4-((8-methylnon-6-enamido)methyl)phenyl 2-((methylamino)methyl)piperidine-1-carboxylate (Compound 1), wherein the pain is reduced by at least 25% compared to placebo. In some embodiments is a method of treating pain in a subject undergoing a bunionectomy comprising administering to the subject 0.5 mg to 5.0 mg of (E)-2-methoxy-4-((8-methylnon-6-enamido)methyl)phenyl 2-((methylamino)methyl)piperidine-1-carboxylate (Compound 1), wherein the pain is reduced by at least 30% compared to placebo.

In another aspect, described herein is a method of treating pain and reducing opioid use in a subject undergoing a bunionectomy, comprising administering to the subject 0.5 mg to 5.0 mg of (E)-2-methoxy-4-((8-methylnon-6-enamido)methyl)phenyl 2-((methylamino)methyl)piperidine-1-carboxylate (Compound 1), wherein opioid use is reduced by at least 20%. In some embodiments is a method of treating pain and reducing opioid use in a subject undergoing a bunionectomy, comprising administering to the subject 0.5 mg to 5.0 mg of (E)-2-methoxy-4-((8-methylnon-6-enamido)methyl)phenyl 2-((methylamino)methyl)piperidine-1-carboxylate (Compound 1), wherein opioid use is reduced by at least 30%. In some embodiments is a method of treating pain and reducing opioid use in a subject undergoing a bunionectomy, comprising administering to the subject 0.5 mg to 5.0 mg of (E)-2-methoxy-4-((8-methylnon-6-enamido)methyl)phenyl 2-((methylamino)methyl)piperidine-1-carboxylate (Compound 1), wherein opioid use is reduced by at least 40%. In some embodiments is a method of treating pain and reducing opioid use in a subject undergoing a bunionectomy, comprising administering to the subject 0.5 mg to 5.0 mg of (E)-2-methoxy-4-((8-methylnon-6-enamido)methyl)phenyl 2-((methylamino)methyl)piperidine-1-carboxylate (Compound 1), wherein opioid use is reduced by at least 50%.

In some embodiments of the methods described herein for use in a subject undergoing a bunionectomy, the subject is administered 2.0 mg to 5.0 mg of (E)-2-methoxy-4-((8-methylnon-6-enamido)methyl)phenyl 2-((methylamino)methyl)piperidine-1-carboxylate. In some embodiments of the methods described herein for use in a subject undergoing a bunionectomy, the subject is administered 3.0 mg to 5.0 mg of (E)-2-methoxy-4-((8-methylnon-6-enamido)methyl)phenyl 2-((methylamino)methyl)piperidine-1-carboxylate. In some embodiments of the methods described herein for use in a subject undergoing a bunionectomy, the subject is administered 4.0 mg to 5.0 mg of (E)-2-methoxy-4-((8-methylnon-6-enamido)methyl)phenyl 2-((methylamino)methyl)piperidine-1-carboxylate.

In another aspect, described herein is a method of treating pain in a subject undergoing a unilateral total knee arthroplasty (TKA) comprising administering to the subject 25 mg to 65 mg of (E)-2-methoxy-4-((8-methylnon-6-enamido)methyl)phenyl 2-((methylamino)methyl)piperidine-1-carboxylate (Compound 1).

In another aspect, described herein is a method of treating pain in a subject undergoing a unilateral total knee arthroplasty (TKA) comprising administering to the subject 25 mg to 65 mg of (E)-2-methoxy-4-((8-methylnon-6-enamido)methyl)phenyl 2-((methylamino)methyl)piperidine-1-carboxylate (Compound 1), wherein the pain is reduced by at least 10% compared to placebo. In some embodiments is a method of treating pain in a subject undergoing a unilateral total knee arthroplasty (TKA) comprising administering to the subject 25 mg to 65 mg of (E)-2-methoxy-4-((8-methylnon-6-enamido)methyl)phenyl 2-((methylamino)methyl)piperidine-1-carboxylate (Compound 1), wherein the pain is reduced by at least 15% compared to placebo. In some embodiments is a method of treating pain in a subject undergoing a unilateral total knee arthroplasty (TKA) comprising administering to the subject 25 mg to 65 mg of (E)-2-methoxy-4-((8-methylnon-6-enamido)methyl)phenyl 2-((methylamino)methyl)piperidine-1-carboxylate (Compound 1), wherein the pain is reduced by at least 20% compared to placebo.

In another aspect, described herein is a method of treating pain and reducing opioid use in a subject undergoing a unilateral total knee arthroplasty (TKA), comprising administering to the subject 25 mg to 65 of (E)-2-methoxy-4-((8-methylnon-6-enamido)methyl)phenyl 2-((methylamino)methyl)piperidine-1-carboxylate (Compound 1), wherein opioid use is reduced by at least 10%. In some embodiments is a method of treating pain and reducing opioid use in a subject undergoing a unilateral total knee arthroplasty (TKA), comprising administering to the subject 25 mg to 65 of (E)-2-methoxy-4-((8-methylnon-6-enamido)methyl)phenyl 2-((methylamino)methyl)piperidine-1-carboxylate (Compound 1), wherein opioid use is reduced by at least 20%. In some embodiments is a method of treating pain and reducing opioid use in a subject undergoing a unilateral total knee arthroplasty (TKA), comprising administering to the subject 25 mg to 65 of (E)-2-methoxy-4-((8-methylnon-6-enamido)methyl)phenyl 2-((methylamino)methyl)piperidine-1-carboxylate (Compound 1), wherein opioid use is reduced by at least 30%.

In some embodiments of the methods described herein for use in a subject undergoing a unilateral total knee arthroplasty (TKA), the subject is administered 25 mg to 50 mg of (E)-2-methoxy-4-((8-methylnon-6-enamido)methyl)phenyl 2-((methylamino)methyl)piperidine-1-carboxylate. In some embodiments of the methods described herein for use in a subject undergoing a unilateral total knee arthroplasty (TKA), the subject is administered 30 mg to 40 mg of (E)-2-methoxy-4-((8-methylnon-6-enamido)methyl)phenyl 2-((methylamino)methyl)piperidine-1-carboxylate.

In another aspect, described herein is a method of treating pain in a subject undergoing laparotomy comprising administering to the subject 25 mg to 65 mg of (E)-2-methoxy-4-((8-methylnon-6-enamido)methyl)phenyl 2-((methylamino)methyl)piperidine-1-carboxylate (Compound 1).

In another aspect, described herein is a method of treating pain in a subject undergoing laparotomy comprising administering to the subject 25 mg to 65 mg of (E)-2-methoxy-4-((8-methylnon-6-enamido)methyl)phenyl 2-((methylamino)methyl)piperidine-1-carboxylate (Compound 1), wherein the pain is reduced by at least 10% compared to placebo. In some embodiments is a method of treating pain in a subject undergoing laparotomy comprising administering to the subject 25 mg to 65 mg of (E)-2-methoxy-4-((8-methylnon-6-enamido)methyl)phenyl 2-((methylamino)methyl)piperidine-1-carboxylate (Compound 1), wherein the pain is reduced by at least 15% compared to placebo. In some embodiments is a method of treating pain in a subject undergoing laparotomy comprising administering to the subject 25 mg to 65 mg of (E)-2-methoxy-4-((8-methylnon-6-enamido)methyl)phenyl 2-((methylamino)methyl)piperidine-1-carboxylate (Compound 1), wherein the pain is reduced by at least 20% compared to placebo.

In another aspect, described herein is a method of treating pain and reducing opioid use in a subject undergoing laparotomy, comprising administering to the subject 25 mg to 65 of (E)-2-methoxy-4-((8-methylnon-6-enamido)methyl)phenyl 2-((methylamino)methyl)piperidine-1-carboxylate (Compound 1), wherein opioid use is reduced by at least 10%. In some embodiments is a method of treating pain and reducing opioid use in a subject undergoing laparotomy, comprising administering to the subject 25 mg to 65 of (E)-2-methoxy-4-((8-methylnon-6-enamido)methyl)phenyl 2-((methylamino)methyl)piperidine-1-carboxylate (Compound 1), wherein opioid use is reduced by at least 20%. In some embodiments is a method of treating pain and reducing opioid use in a subject undergoing laparotomy, comprising administering to the subject 25 mg to 65 of (E)-2-methoxy-4-((8-methylnon-6-enamido)methyl)phenyl 2-((methylamino)methyl)piperidine-1-carboxylate (Compound 1), wherein opioid use is reduced by at least 30%. In some embodiments of the methods described herein for use in a subject undergoing laparotomy, the subject is administered 25 mg to 50 mg of (E)-2-methoxy-4-((8-methylnon-6-enamido)methyl)phenyl 2-((methylamino)methyl)piperidine-1-carboxylate. In some embodiments of the methods described herein for use in a subject undergoing laparotomy, the subject is administered 30 mg to 40 mg of (E)-2-methoxy-4-((8-methylnon-6-enamido)methyl)phenyl 2-((methylamino)methyl)piperidine-1-carboxylate. In some embodiments, the subject is undergoing laparotomy to repair a ventral hernia.

In some embodiments of the methods described herein, (E)-2-methoxy-4-((8-methylnon-6-enamido)methyl)phenyl 2-((methylamino)methyl)piperidine-1-carboxylate (Compound 1) is administered by infiltration and instillation. In some embodiments of the methods described herein, 10% to 35% of (E)-2-methoxy-4-((8-methylnon-6-enamido)methyl)phenyl 2-((methylamino)methyl)piperidine-1-carboxylate (Compound 1) is administered by instillation and 65% to 90% of (E)-2-methoxy-4-((8-methylnon-6-enamido)methyl)phenyl 2-((methylamino)methyl)piperidine-1-carboxylate (Compound 1) is administered by infiltration. In some embodiments of the methods described herein, 15% to 30% of (E)-2-methoxy-4-((8-methylnon-6-enamido)methyl)phenyl 2-((methylamino)methyl)piperidine-1-carboxylate (Compound 1) is administered by instillation and 70% to 85% of (E)-2-methoxy-4-((8-methylnon-6-enamido)methyl)phenyl 2-((methylamino)methyl)piperidine-1-carboxylate (Compound 1) is administered by infiltration.

### BREIF DESCRIPTION OF THE DRAWINGS

FIG. 1 shows the bunionectomy Phase 2 clinical study design.

FIG. 2 shows the dose response in pain reduction for three dosing groups of Compound 1 (0.7 mg, 2.1 mg, and 4.2 mg) in the bunionectomy clinical study.

FIG. 3 shows the reduction in opioid consumption for three dosing groups of Compound 1 (0.7 mg, 2.1 mg, and 4.2 mg) in the bunionectomy clinical study.

FIG. 4 shows the analgesic effect to Day 15 for bunionectomy patients dosed at 4.2 mg of Compound 1 in the bunionectomy clinical study.

FIG. 5 shows the unilateral total knee arthroplasty (TKA) Phase 1/2 clinical study design (Part A).

FIG. 6 shows the unilateral total knee arthroplasty (TKA) Phase 1/2 clinical study design (Part B).

FIG. 7 shows the pain reduction for two dosing groups of Compound 1 (36 mg and 60 mg) in the unilateral total knee arthroplasty (TKA) clinical study.

FIG. 8 shows the reduction in opioid consumption for two dosing groups of Compound 1 (36 mg and 60 mg) in the unilateral total knee arthroplasty (TKA) clinical study.

FIG. 9 shows the reduction in pain and opioid consumption in patients treated with Compound 1 (36 mg) compared to placebo in the laparotomy for ventral hernia repair clinical study.

### DETAILED DESCRIPTION

Capsaicin is a transient receptor potential cation channel, subfamily V (vanilloid), member 1 (TRPV1) agonist. TRPV1 is a ligand-gated, nonselective, cation channel preferentially expressed most densely in C-fiber nociceptors and to a lesser extent on A6—fiber nociceptors (Babbar 2009, Caterina 2001). TRPV1 responds to noxious stimuli including capsaicin, heat, and extracellular acidification, and integrates simultaneous exposures to these stimuli (Suresh 2010, Surh 1995, Tominaga 1998).

Capsaicin exposure to TRPV-1-expressing nociceptor peripheral terminals results in initial excitation of the nociceptor followed by a functional desensitization which continues for some time after removal of capsaicin from the site. Capsaicin, however, is virtually insoluble in aqueous media or local anesthetic solutions which means that capsaicin formulations tend to be quite hydrophobic and viscous making them hard to inject and less likely to permeate surgical site tissues.

To overcome the solubility limitations of capsaicin, the highly water-soluble capsaicin pro-drug (E)-2-methoxy-4-((8-methylnon-6-enamido)methyl)phenyl 2-((methylamino)methyl)piperidine-1-carboxylate (Compound 1) was developed for local infiltration. It avoids the physicochemical limitations of capsaicin while providing greater target engagement which theoretically would produce superior local analgesia, particularly after surgical trauma. Based upon this mechanism of action, local delivery of a TRPV1 agonist throughout the tissues around the surgical site prior to wound closure to maximize target engagement should result in a meaningful reduction of post-surgical pain over several days to weeks. This improved long-term pain relief has the ability to augment current multimodal analgesia or enhanced recovery programs which may help to avoid the need for supplemental opioid use after surgery.

### Certain Terminology

Unless defined otherwise, all technical and scientific terms used herein have the same meaning as is commonly understood to which the claimed subject matter belongs. In the event that there are a plurality of definitions for terms herein, those in this section prevail. All patents, patent applications, publications, and published nucleotide and amino acid sequences (e.g., sequences available in GenBank or other databases) referred to herein are incorporated by reference. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include,” “includes,” and “included,” is not limiting. The term “about” when referring to a number or a numerical range means that the number or numerical range referred to is an approximation within experimental variability (or within statistical experimental error), and thus the number or numerical range varies between 1% and 15% of the stated number or numerical range.

The section headings used herein are for organizational purposes only and are not to be construed as limiting the subject matter described.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods, compounds, and compositions described herein.

The terms “kit” and “article of manufacture” are used as synonyms.

The term “subject” or “patient” encompasses mammals and non-mammals. Examples of mammals include, but are not limited to, any member of the Mammalian class: humans, non- human primates such as chimpanzees, and other apes and monkey species; farm animals such as cattle, horses, sheep, goats, and swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” as used herein, include alleviating, abating or ameliorating a disease or condition symptoms, preventing additional symptoms, ameliorating or preventing the underlying causes of symptoms, inhibiting the disease or condition (e.g., arresting the development of the disease or condition), relieving the disease or condition, causing regression of the disease or condition, relieving a condition caused by the disease or condition, or stopping the symptoms of the disease or condition either prophylactically and/or therapeutically. In some embodiments, the methods of treating pain in a subject undergoing surgery described herein are equivalent to methods of treating surgical pain in a subj ect.

As used herein, amelioration of the symptoms of a particular disease, disorder, or condition by administration of a particular compound or pharmaceutical composition refers to any lessening of severity, delay in onset, slowing of progression, or shortening of duration, whether permanent or temporary, lasting or transient that can be attributed to or associated with administration of the compound or composition.

The term “modulate,” as used herein, means to interact with a target protein either directly or indirectly so as to alter the activity of the target protein, including, by way of example only, to inhibit the activity of the target, or to limit or reduce the activity of the target.

As used herein, the term “modulator” refers to a compound that alters an activity of a target. For example, a modulator can cause an increase or decrease in the magnitude of a certain activity of a target compared to the magnitude of the activity in the absence of the modulator. In certain embodiments, a modulator is an inhibitor, which decreases the magnitude of one or more activities of a target. In certain embodiments, an inhibitor completely prevents one or more activities of a target.

The term “acceptable” with respect to a formulation, composition or ingredient, as used herein, means having no persistent detrimental effect on the general health of the subject being treated.

By “pharmaceutically acceptable,” as used herein, refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutical combination” as used herein, means a product that results from the mixing or combining of more than one active ingredient and includes both fixed and non-fixed combinations of the active ingredients. The term “fixed combination” means that one active ingredient, e.g compound described herein, and a co-agent, are both administered to a patient simultaneously in the form of a single entity or dosage. The term “non-fixed combination” means that one active ingredient, e.g. a compound decribed herein and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “pharmaceutical composition” refers to a mixture of a compound described herein with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the pharmaceutical composition that includes a compound described herein required to provide a clinically significant decrease in disease symptoms. An appropriate “effective” amount in any individual case may be determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “co-administration” or the like, as used herein, are meant to encompass administration of the selected therapeutic agents to a single patient, and are intended to include treatment regimens in which the agents are administered by the same or different route of administration or at the same or different time.

The term “carrier,” as used herein, refers to relatively nontoxic chemical compounds or agents that facilitate the incorporation of a compound into cells or tissues.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Further information on metabolism may be obtained from The Pharmacological Basis of Therapeutics, 9th Edition, McGraw-Hill (1996). Metabolites of the compounds disclosed herein can be identified either by administration of compounds to a host and analysis of tissue samples from the host, or by incubation of compounds with hepatic cells in vitro and analysis of the resulting compounds.

“Bioavailability” refers to the percentage of the weight of the compound disclosed herein that is delivered into the general circulation of the animal or human being studied. The total exposure (AUC(0-∞)) of a drug when administered intravenously is usually defined as 100% bioavailable (F%). “Oral bioavailability” refers to the extent to which a compound disclosed herein, is absorbed into the general circulation when the pharmaceutical composition is taken orally as compared to intravenous injection.

“Blood plasma concentration” refers to the concentration of a compound disclosed herein, in the plasma component of blood of a subject. It is understood that the plasma concentration of compounds described herein may vary significantly between subjects, due to variability with respect to metabolism and/or possible interactions with other therapeutic agents. In accordance with one embodiment disclosed herein, the blood plasma concentration of the compounds disclosed herein may vary from subject to subject. Likewise, values such as maximum plasma concentration (Cmax) or time to reach maximum plasma concentration (Tmax), or total area under the plasma concentration time curve (AUC(0-∞)) may vary from subject to subject. Due to this variability, the amount necessary to constitute “a therapeutically effective amount” of a compound may vary from subject to subject.

“Blood concentration” refers to the concentration of a compound disclosed herein, in the blood of a subject. It is understood that the blood concentration of compounds described herein may vary significantly between subjects, due to variability with respect to metabolism and/or possible interactions with other therapeutic agents. In accordance with one embodiment disclosed herein, the blood concentration of the compounds disclosed herein may vary from subject to subject. Likewise, values such as maximum blood concentration (Cmax) or time to reach maximum blood concentration (Tmax), or total area under the blood concentration time curve (AUC_{(0-∞)}) may vary from subject to subject. Due to this variability, the amount necessary to constitute “a therapeutically effective amount” of a compound may vary from subject to subject.

### Compound 1

The chemical structure of Compound 1 ((E)-2-methoxy-4-((8-methylnon-6-enamido)methyl)phenyl 2-((methylamino)methyl)piperidine-1-carboxylate) is shown below:

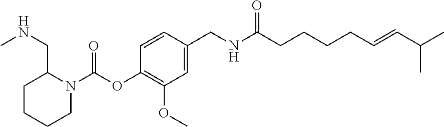 [SMILES: CNCC1CCCCN1C(=O)Oc1ccc(CNC(=O)CCCC/C=C/C(C)C)cc1OC]

Compound 1 releases capsaicin and cyclic urea Compound 2 (2-methylhexahydroimidazo[1,5-a]pyridin-3(2H)-one) under well-defined rates via a pH driven, intra-molecular cyclization release reaction after Compound 1 has been delivered to the body and/or is exposed to specific physiological conditions:

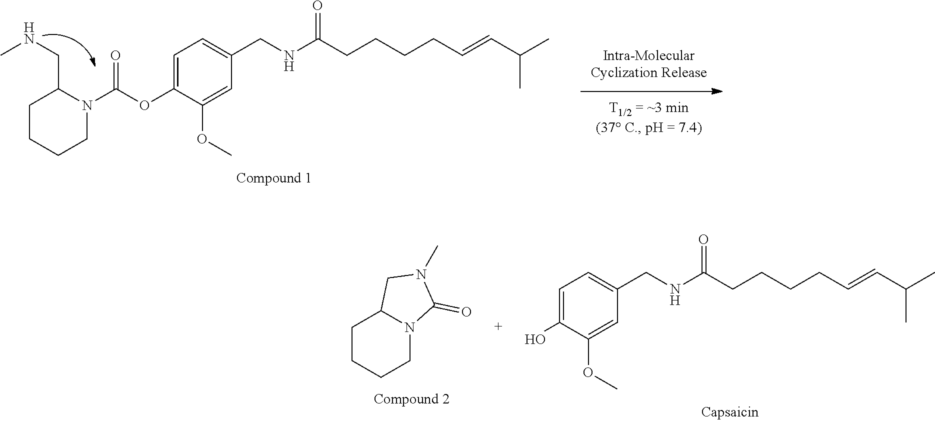 [SMILES: CNCC1CCCCN1C(=O)Oc1ccc(CNC(=O)CCCC/C=C/C(C)C)cc1OC ; CN1CC2CCCCN2C1=O ; COc1cc(CNC(=O)CCCC/C=C/C(C)C)ccc1O]

The preparation of Compound 1 was disclosed in US 2016/0145225, the content of which is incorporated by reference in its entirety.

In some embodiments, (E)-2-methoxy-4-((8-methylnon-6-enamido)methyl)phenyl 2-((methylamino)methyl)piperidine-1-carboxylate (Compound 1) is in a pharmaceutically acceptable salt form. In some embodiments, a pharmaceutically acceptable salt of (E)-2-methoxy-4-((8-methylnon-6-enamido)methyl)phenyl 2-((methylamino)methyl)piperidine-1-carboxylate (Compound 1) is a hydrochloride salt. Methods

In some embodiments described herein is a method of treating pain in a subject undergoing surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.05 mg/mL to 0.5 mg/mL. In some embodiments is a method of treating pain in a subject undergoing surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.1 mg/mL to 0.5 mg/mL. In some embodiments is a method of treating pain in a subject undergoing surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.1 mg/mL to 0.45 mg/mL. In some embodiments is a method of treating pain in a subject undergoing surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.1 mg/mL to 0.4 mg/mL. In some embodiments is a method of treating pain in a subject undergoing surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.1 mg/mL to 0.35 mg/mL. In some embodiments is a method of treating pain in a subject undergoing surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.15 mg/mL to 0.4 mg/mL. In some embodiments is a method of treating pain in a subject undergoing surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.15 mg/mL to 0.35 mg/mL. In some embodiments is a method of treating pain in a subject undergoing surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.2 mg/mL to 0.35 mg/mL. In some embodiments is a method of treating pain in a subject undergoing surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.15 mg/mL to 0.3 mg/mL. In some embodiments is a method of treating pain in a subject undergoing surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.2 mg/mL to 0.3 mg/mL. In some embodiments is a method of treating pain in a subject undergoing surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.5 mg/mL. In some embodiments is a method of treating pain in a subject undergoing surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.45 mg/mL. In some embodiments is a method of treating pain in a subject undergoing surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.4 mg/mL. In some embodiments is a method of treating pain in a subject undergoing surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.35 mg/mL. In some embodiments is a method of treating pain in a subject undergoing surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.3 mg/mL. In some embodiments is a method of treating pain in a subject undergoing surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.25 mg/mL. In some embodiments is a method of treating pain in a subject undergoing surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.2 mg/mL. In some embodiments is a method of treating pain in a subject undergoing surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.15 mg/mL. In some embodiments is a method of treating pain in a subject undergoing surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.1 mg/mL. In some embodiments is a method of treating pain in a subject undergoing surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.05 mg/mL. In some embodiments, the pain is from orthopedic surgery. In some embodiments, the pain is from a bunionectomy. In some embodiments, the pain is from a unilateral total knee arthroplasty (TKA). In some embodiments, the pain is from an abdominal incision. In some embodiments, the pain is from an abdominoplasty. In some embodiments, the pain is from repair of an inguinal hernia. In some embodiments, the pain is from a laparotomy. In some embodiments, the pain is from a laparotomy selected from a laparotomy to repair a ventral hernia, a C-section, hysterectomy, intestinal resection, and nephrectomy. In some embodiments, the pain is from a laparotomy to repair a ventral hernia. In some embodiments, the pain is from a C-section. In some embodiments, the pain is from a hysterectomy. In some embodiments, the pain is from an intestinal resection. In some embodiments, the pain is from a nephrectomy. In some embodiments, the pain is from general surgery. In some embodiments, the pain is from obstetric and gynecological surgery. In some embodiments, the pain is from plastic surgery.

In some embodiments described herein is a method of treating pain in a subject undergoing orthopedic surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.05 mg/mL to 0.5 mg/mL. In some embodiments is a method of treating pain in a subject undergoing orthopedic surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.1 mg/mL to 0.5 mg/mL. In some embodiments is a method of treating pain in a subject undergoing orthopedic surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.1 mg/mL to 0.45 mg/mL. In some embodiments is a method of treating pain in a subject undergoing orthopedic surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.1 mg/mL to 0.4 mg/mL. In some embodiments is a method of treating pain in a subject undergoing orthopedic surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.1 mg/mL to 0.35 mg/mL. In some embodiments is a method of treating pain in a subject undergoing orthopedic surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.15 mg/mL to 0.4 mg/mL. In some embodiments is a method of treating pain in a subject undergoing orthopedic surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.15 mg/mL to 0.35 mg/mL. In some embodiments is a method of treating pain in a subject undergoing orthopedic surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.2 mg/mL to 0.35 mg/mL. In some embodiments is a method of treating pain in a subject undergoing orthopedic surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.15 mg/mL to 0.3 mg/mL. In some embodiments is a method of treating pain in a subject undergoing orthopedic surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.2 mg/mL to 0.3 mg/mL. In some embodiments is a method of treating pain in a subject undergoing orthopedic surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.5 mg/mL. In some embodiments is a method of treating pain in a subject undergoing orthopedic surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.45 mg/mL. In some embodiments is a method of treating pain in a subject undergoing orthopedic surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.4 mg/mL. In some embodiments is a method of treating pain in a subject undergoing orthopedic surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.35 mg/mL. In some embodiments is a method of treating pain in a subject undergoing orthopedic surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.3 mg/mL. In some embodiments is a method of treating pain in a subject undergoing orthopedic surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.25 mg/mL. In some embodiments is a method of treating pain in a subject undergoing orthopedic surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.2 mg/mL. In some embodiments is a method of treating pain in a subject undergoing orthopedic surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.15 mg/mL. In some embodiments is a method of treating pain in a subject undergoing orthopedic surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.1 mg/mL. In some embodiments is a method of treating pain in a subject undergoing orthopedic surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.05 mg/mL.

In some embodiments described herein is a method of treating pain in a subject undergoing a bunionectomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.05 mg/mL to 0.5 mg/mL. In some embodiments is a method of treating pain in a subject undergoing a bunionectomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.1 mg/mL to 0.5 mg/mL. In some embodiments is a method of treating pain in a subject undergoing a bunionectomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.1 mg/mL to 0.45 mg/mL. In some embodiments is a method of treating pain in a subject undergoing a bunionectomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.1 mg/mL to 0.4 mg/mL. In some embodiments is a method of treating pain in a subject undergoing a bunionectomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.1 mg/mL to 0.35 mg/mL. In some embodiments is a method of treating pain in a subject undergoing a bunionectomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.15 mg/mL to 0.4 mg/mL. In some embodiments is a method of treating pain in a subject undergoing a bunionectomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.15 mg/mL to 0.35 mg/mL. In some embodiments is a method of treating pain in a subject undergoing a bunionectomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.2 mg/mL to 0.35 mg/mL. In some embodiments is a method of treating pain in a subject undergoing a bunionectomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.15 mg/mL to 0.3 mg/mL. In some embodiments is a method of treating pain in a subject undergoing a bunionectomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.2 mg/mL to 0.3 mg/mL. In some embodiments is a method of treating pain in a subject undergoing a bunionectomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.5 mg/mL. In some embodiments is a method of treating pain in a subject undergoing a bunionectomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.45 mg/mL. In some embodiments is a method of treating pain in a subject undergoing a bunionectomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.4 mg/mL. In some embodiments is a method of treating pain in a subject undergoing a bunionectomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.35 mg/mL. In some embodiments is a method of treating pain in a subject undergoing a bunionectomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.3 mg/mL. In some embodiments is a method of treating pain in a subject undergoing a bunionectomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.25 mg/mL. In some embodiments is a method of treating pain in a subject undergoing a bunionectomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.2 mg/mL. In some embodiments is a method of treating pain in a subject undergoing a bunionectomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.15 mg/mL. In some embodiments is a method of treating pain in a subject undergoing a bunionectomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.1 mg/mL. In some embodiments is a method of treating pain in a subject undergoing a bunionectomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.05 mg/mL.

In some embodiments described herein is a method of treating pain in a subject undergoing a unilateral total knee arthroplasty (TKA) comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.05 mg/mL to 0.5 mg/mL. In some embodiments is a method of treating pain in a subject undergoing a unilateral total knee arthroplasty (TKA) comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.1 mg/mL to 0.5 mg/mL. In some embodiments is a method of treating pain in a subject undergoing a unilateral total knee arthroplasty (TKA) comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.1 mg/mL to 0.45 mg/mL. In some embodiments is a method of treating pain in a subject undergoing a unilateral total knee arthroplasty (TKA) comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.1 mg/mL to 0.4 mg/mL. In some embodiments is a method of treating pain in a subject undergoing a unilateral total knee arthroplasty (TKA) comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.1 mg/mL to 0.35 mg/mL. In some embodiments is a method of treating pain in a subject undergoing a unilateral total knee arthroplasty (TKA) comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.15 mg/mL to 0.4 mg/mL. In some embodiments is a method of treating pain in a subject undergoing a unilateral total knee arthroplasty (TKA) comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.15 mg/mL to 0.35 mg/mL. In some embodiments is a method of treating pain in a subject undergoing a unilateral total knee arthroplasty (TKA) comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.2 mg/mL to 0.35 mg/mL. In some embodiments is a method of treating pain in a subject undergoing a unilateral total knee arthroplasty (TKA) comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.15 mg/mL to 0.3 mg/mL. In some embodiments is a method of treating pain in a subject undergoing a unilateral total knee arthroplasty (TKA) comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.2 mg/mL to 0.3 mg/mL. In some embodiments is a method of treating pain in a subject undergoing a unilateral total knee arthroplasty (TKA) comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.5 mg/mL. In some embodiments is a method of treating pain in a subject undergoing a unilateral total knee arthroplasty (TKA) comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.45 mg/mL. In some embodiments is a method of treating pain in a subject undergoing a unilateral total knee arthroplasty (TKA) comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.4 mg/mL. In some embodiments is a method of treating pain in a subject undergoing a unilateral total knee arthroplasty (TKA) comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.35 mg/mL. In some embodiments is a method of treating pain in a subject undergoing a unilateral total knee arthroplasty (TKA) comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.3 mg/mL. In some embodiments is a method of treating pain in a subject undergoing a unilateral total knee arthroplasty (TKA) comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.25 mg/mL. In some embodiments is a method of treating pain in a subject undergoing a unilateral total knee arthroplasty (TKA) comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.2 mg/mL. In some embodiments is a method of treating pain in a subject undergoing a unilateral total knee arthroplasty (TKA) comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.15 mg/mL. In some embodiments is a method of treating pain in a subject undergoing a unilateral total knee arthroplasty (TKA) comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.1 mg/mL. In some embodiments is a method of treating pain in a subject undergoing a unilateral total knee arthroplasty (TKA) comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.05 mg/mL.

In some embodiments described herein is a method of treating pain in a subject undergoing laparotomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.05 mg/mL to 0.5 mg/mL. In some embodiments is a method of treating pain in a subject undergoing laparotomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.1 mg/mL to 0.5 mg/mL. In some embodiments is a method of treating pain in a subject undergoing laparotomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.1 mg/mL to 0.45 mg/mL. In some embodiments is a method of treating pain in a subject undergoing laparotomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.1 mg/mL to 0.4 mg/mL. In some embodiments is a method of treating pain in a subject undergoing laparotomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.1 mg/mL to 0.35 mg/mL. In some embodiments is a method of treating pain in a subject undergoing laparotomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.15 mg/mL to 0.4 mg/mL. In some embodiments is a method of treating pain in a subject undergoing lapartomy to repair a ventral hernia comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.15 mg/mL to 0.35 mg/mL. In some embodiments is a method of treating pain in a subject undergoing laparotomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.2 mg/mL to 0.35 mg/mL. In some embodiments is a method of treating pain in a subject undergoing laparotomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.15 mg/mL to 0.3 mg/mL. In some embodiments is a method of treating pain in a subject undergoing laparotomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.2 mg/mL to 0.3 mg/mL. In some embodiments is a method of treating pain in a subject undergoing laparotomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.5 mg/mL. In some embodiments is a method of treating pain in a subject undergoing laparotomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.45 mg/mL. In some embodiments is a method of treating pain in a subject undergoing laparotomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.4 mg/mL. In some embodiments is a method of treating pain in a subject undergoing laparotomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.35 mg/mL. In some embodiments is a method of treating pain in a subject undergoing laparotomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.3 mg/mL. In some embodiments is a method of treating pain in a subject undergoing laparotomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.25 mg/mL. In some embodiments is a method of treating pain in a subject undergoing laparotomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.2 mg/mL. In some embodiments is a method of treating pain in a subject undergoing laparotomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.15 mg/mL. In some embodiments is a method of treating pain in a subject undergoing laparotomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.1 mg/mL. In some embodiments is a method of treating pain in a subject undergoing laparotomy comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.05 mg/mL.

In some embodiments of the methods described herein, the laparotomy is selected from a laparotomy to repair a ventral hernia, a C-section, hysterectomy, intestinal resection, and nephrectomy.

In some embodiments described herein is a method of treating pain in a subject undergoing laparotomy to repair a ventral hernia comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.05 mg/mL to 0.5 mg/mL. In some embodiments is a method of treating pain in a subject undergoing laparotomy to repair a ventral hernia comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.1 mg/mL to 0.5 mg/mL. In some embodiments is a method of treating pain in a subject undergoing laparotomy to repair a ventral hernia comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.1 mg/mL to 0.45 mg/mL. In some embodiments is a method of treating pain in a subject undergoing laparotomy to repair a ventral hernia comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.1 mg/mL to 0.4 mg/mL. In some embodiments is a method of treating pain in a subject undergoing laparotomy to repair a ventral hernia comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.1 mg/mL to 0.35 mg/mL. In some embodiments is a method of treating pain in a subject undergoing laparotomy to repair a ventral hernia comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.15 mg/mL to 0.4 mg/mL. In some embodiments is a method of treating pain in a subject undergoing lapartomy to repair a ventral hernia comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.15 mg/mL to 0.35 mg/mL. In some embodiments is a method of treating pain in a subject undergoing laparotomy to repair a ventral hernia comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.2 mg/mL to 0.35 mg/mL. In some embodiments is a method of treating pain in a subject undergoing laparotomy to repair a ventral hernia comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.15 mg/mL to 0.3 mg/mL. In some embodiments is a method of treating pain in a subject undergoing laparotomy to repair a ventral hernia comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.2 mg/mL to 0.3 mg/mL. In some embodiments is a method of treating pain in a subject undergoing laparotomy to repair a ventral hernia comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.5 mg/mL. In some embodiments is a method of treating pain in a subject undergoing laparotomy to repair a ventral hernia comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.45 mg/mL. In some embodiments is a method of treating pain in a subject undergoing laparotomy to repair a ventral hernia comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.4 mg/mL. In some embodiments is a method of treating pain in a subject undergoing laparotomy to repair a ventral hernia comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.35 mg/mL. In some embodiments is a method of treating pain in a subject undergoing laparotomy to repair a ventral hernia comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.3 mg/mL. In some embodiments is a method of treating pain in a subject undergoing laparotomy to repair a ventral hernia comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.25 mg/mL. In some embodiments is a method of treating pain in a subject undergoing laparotomy to repair a ventral hernia comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.2 mg/mL. In some embodiments is a method of treating pain in a subject undergoing laparotomy to repair a ventral hernia comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.15 mg/mL. In some embodiments is a method of treating pain in a subject undergoing laparotomy to repair a ventral hernia comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.1 mg/mL. In some embodiments is a method of treating pain in a subject undergoing laparotomy to repair a ventral hernia comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of about 0.05 mg/mL.

In some embodiments described herein is a method of treating pain in a subject undergoing a bunionectomy comprising administering to the subject 0.5 mg to 5.0 mg of (E)-2-methoxy-4-((8-methylnon-6-enamido)methyl)phenyl 2-((methylamino)methyl)piperidine-1-carboxylate (Compound 1). In some embodiments described herein is a method of treating pain in a subject undergoing a bunionectomy comprising administering to the subject 1.0 mg to 5.0 mg of (E)-2-methoxy-4-((8-methylnon-6-enamido)methyl)phenyl 2-((methylamino)methyl)piperidine-1-carboxylate (Compound 1). In some embodiments described herein is a method of treating pain in a subject undergoing a bunionectomy comprising administering to the subject 1.5 mg to 5.0 mg of (E)-2-methoxy-4-((8-methylnon-6-enamido)methyl)phenyl 2-((methylamino)methyl)piperidine-1-carboxylate (Compound 1). In some embodiments described herein is a method of treating pain in a subject undergoing a bunionectomy comprising administering to the subject 2.0 mg to 5.0 mg of (E)-2-methoxy-4-((8-methylnon-6-enamido)methyl)phenyl 2-((methylamino)methyl)piperidine-1-carboxylate (Compound 1). In some embodiments described herein is a method of treating pain in a subject undergoing a bunionectomy comprising administering to the subject 2.5 mg to 5.0 mg of (E)-2-methoxy-4-((8-methylnon-6-enamido)methyl)phenyl 2-((methylamino)methyl)piperidine-1-carboxylate (Compound 1). In some embodiments described herein is a method of treating pain in a subject undergoing a bunionectomy comprising administering to the subject 3.0 mg to 5.0 mg of (E)-2-methoxy-4-((8-methylnon-6-enamido)methyl)phenyl 2-((methylamino)methyl)piperidine-1-carboxylate (Compound 1). In some embodiments described herein is a method of treating pain in a subject undergoing a bunionectomy comprising administering to the subject 3.5 mg to 5.0 mg of (E)-2-methoxy-4-((8-methylnon-6-enamido)methyl)phenyl 2-((methylamino)methyl)piperidine-1-carboxylate (Compound 1). In some embodiments described herein is a method of treating pain in a subject undergoing a bunionectomy comprising administering to the subject 4.0 mg to 5.0 mg of (E)-2-methoxy-4-((8-methylnon-6-enamido)methyl)phenyl 2-((methylamino)methyl)piperidine-1-carboxylate (Compound 1). In some embodiments described herein is a method of treating pain in a subject undergoing a bunionectomy comprising administering to the subject 4.0 mg to 4.5 mg of (E)-2-methoxy-4-((8-methylnon-6-enamido)methyl)phenyl 2-((methylamino)methyl)piperidine-1-carboxylate (Compound 1). In some embodiments described herein is a method of treating pain in a subject undergoing a bunionectomy comprising administering to the subject about 5.0 mg of (E)-2-methoxy-4-((8-methylnon-6-enamido)methyl)phenyl 2-((methylamino)methyl)piperidine-1-carboxylate (Compound 1). In some embodiments described herein is a method of treating pain in a subject undergoing a bunionectomy comprising administering to the subject about 4.5 mg of (E)-2-methoxy-4-((8-methylnon-6-enamido)methyl)phenyl 2-((methylamino)methyl)piperidine-1-carboxylate (Compound 1). In some embodiments described herein is a method of treating pain in a subject undergoing a bunionectomy comprising administering to the subject about 4.2 mg of (E)-2-methoxy-4-((8-methylnon-6-enamido)methyl)phenyl 2-((methylamino)methyl)piperidine-1-carboxylate (Compound 1). In some embodiments described herein is a method of treating pain in a subject undergoing a bunionectomy comprising administering to the subject about 4.0 mg of (E)-2-methoxy-4-((8-methylnon-6-enamido)methyl)phenyl 2-((methylamino)methyl)piperidine-1-carboxylate (Compound 1). In some embodiments described herein is a method of treating pain in a subject undergoing a bunionectomy comprising administering to the subject about 3.5 mg of (E)-2-methoxy-4-((8-methylnon-6-enamido)methyl)phenyl 2-((methylamino)methyl)piperidine-1-carboxylate (Compound 1). In some embodiments described herein is a method of treating pain in a subject undergoing a bunionectomy comprising administering to the subject about 3.0 mg of (E)-2-methoxy-4-((8-methylnon-6-enamido)methyl)phenyl 2-((methylamino)methyl)piperidine-1-carboxylate (Compound 1). In some embodiments described herein is a method of treating pain in a subject undergoing a bunionectomy comprising administering to the subject about 2.5 mg of (E)-2-methoxy-4-((8-methylnon-6-enamido)methyl)phenyl 2-((methylamino)methyl)piperidine-1-carboxylate (Compound 1). In some embodiments described herein is a method of treating pain in a subject undergoing a bunionectomy comprising administering to the subject about 2.0 mg of (E)-2-methoxy-4-((8-methylnon-6-enamido)methyl)phenyl 2-((methylamino)methyl)piperidine-1-carboxylate (Compound 1). In some embodiments described herein is a method of treating pain in a subject undergoing a bunionectomy comprising administering to the subject about 1.5 mg of (E)-2-methoxy-4-((8-methylnon-6-enamido)methyl)phenyl 2-((methylamino)methyl)piperidine-1-carboxylate (Compound 1). In some embodiments described herein is a method of treating pain in a subject undergoing a bunionectomy comprising administering to the subject about 1.0 mg of (E)-2-methoxy-4-((8-methylnon-6-enamido)methyl)phenyl 2-((methylamino)methyl)piperidine-1-carboxylate (Compound 1). In some embodiments described herein is a method of treating pain in a subject undergoing a bunionectomy comprising administering to the subject about 0.5 mg of (E)-2-methoxy-4-((8-methylnon-6-enamido)methyl)phenyl 2-((methylamino)methyl)piperidine-1-carboxylate (Compound 1).

In some embodiments described herein is a method of treating pain in a subject undergoing a bunionectomy comprising administering to the subject 0.5 mg to 5.0 mg of (E)-2-methoxy-4-((8-methylnon-6-enamido)methyl)phenyl 2-((methylamino)methyl)piperidine-1-carboxylate (Compound 1), wherein the pain is reduced by at least 15% compared to placebo. In some embodiments is a method of treating pain in a subject undergoing a bunionectomy comprising administering to the subject 0.5 mg to 5.0 mg of (E)-2-methoxy-4-((8-methylnon-6-enamido)methyl)phenyl 2-((methylamino)methyl)piperidine-1-carboxylate (Compound 1), wherein the pain is reduced by at least 20% compared to placebo. In some embodiments is a method of treating pain in a subject undergoing a bunionectomy comprising administering to the subject 0.5 mg to 5.0 mg of (E)-2-methoxy-4-((8-methylnon-6-enamido)methyl)phenyl 2-((methylamino)methyl)piperidine-1-carboxylate (Compound 1), wherein the pain is reduced by at least 25% compared to placebo. In some embodiments is a method of treating pain in a subject undergoing a bunionectomy comprising administering to the subject 0.5 mg to 5.0 mg of (E)-2-methoxy-4-((8-methylnon-6-enamido)methyl)phenyl 2-((methylamino)methyl)piperidine-1-carboxylate (Compound 1), wherein the pain is reduced by at least 30% compared to placebo.

In some embodiments, described herein is a method of treating pain and reducing opioid use in a subject undergoing a bunionectomy, comprising administering to the subject 0.5 mg to 5.0 mg of (E)-2-methoxy-4-((8-methylnon-6-enamido)methyl)phenyl 2-((methylamino)methyl)piperidine-1-carboxylate (Compound 1), wherein opioid use is reduced by at least 20%. In some embodiments is a method of treating pain and reducing opioid use in a subject undergoing a bunionectomy, comprising administering to the subject 0.5 mg to 5.0 mg of (E)-2-methoxy-4-((8-methylnon-6-enamido)methyl)phenyl 2-((methylamino)methyl)piperidine-1-carboxylate (Compound 1), wherein opioid use is reduced by at least 25%. In some embodiments is a method of treating pain and reducing opioid use in a subject undergoing a bunionectomy, comprising administering to the subject 0.5 mg to 5.0 mg of (E)-2-methoxy-4-((8-methylnon-6-enamido)methyl)phenyl 2-((methylamino)methyl)piperidine-1-carboxylate (Compound 1), wherein opioid use is reduced by at least 30%. In some embodiments is a method of treating pain and reducing opioid use in a subject undergoing a bunionectomy, comprising administering to the subject 0.5 mg to 5.0 mg of (E)-2-methoxy-4-((8-methylnon-6-enamido)methyl)phenyl 2-((methylamino)methyl)piperidine-1-carboxylate (Compound 1), wherein opioid use is reduced by at least 35%. In some embodiments is a method of treating pain and reducing opioid use in a subject undergoing a bunionectomy, comprising administering to the subject 0.5 mg to 5.0 mg of (E)-2-methoxy-4-((8-methylnon-6-enamido)methyl)phenyl 2-((methylamino)methyl)piperidine-1-carboxylate (Compound 1), wherein opioid use is reduced by at least 40%. In some embodiments is a method of treating pain and reducing opioid use in a subject undergoing a bunionectomy, comprising administering to the subject 0.5 mg to 5.0 mg of (E)-2-methoxy-4-((8-methylnon-6-enamido)methyl)phenyl 2-((methylamino)methyl)piperidine-1-carboxylate (Compound 1), wherein opioid use is reduced by at least 45%. In some embodiments is a method of treating pain and reducing opioid use in a subject undergoing a bunionectomy, comprising administering to the subject 0.5 mg to 5.0 mg of (E)-2-methoxy-4-((8-methylnon-6-enamido)methyl)phenyl 2-((methylamino)methyl)piperidine-1-carboxylate (Compound 1), wherein opioid use is reduced by at least 50%.

In some embodiments of the methods described herein for use in a subject undergoing a bunionectomy, the subject is administered 1.0 mg to 5.0 mg of (E)-2-methoxy-4-((8-methylnon-6-enamido)methyl)phenyl 2-((methylamino)methyl)piperidine-1-carboxylate. In some embodiments of the methods described herein for use in a subject undergoing a bunionectomy, the subject is administered 2.0 mg to 5.0 mg of (E)-2-methoxy-4-((8-methylnon-6-enamido)methyl)phenyl 2-((methylamino)methyl)piperidine-1-carboxylate. In some embodiments of the methods described herein for use in a subject undergoing a bunionectomy, the subject is administered 3.0 mg to 5.0 mg of (E)-2-methoxy-4-((8-methylnon-6-enamido)methyl)phenyl 2-((methylamino)methyl)piperidine-1-carboxylate. In some embodiments of the methods described herein for use in a subject undergoing a bunionectomy, the subject is administered 4.0 mg to 5.0 mg of (E)-2-methoxy-4-((8-methylnon-6-enamido)methyl)phenyl 2-((methylamino)methyl)piperidine-1-carboxylate. In some embodiments of the methods described herein for use in a subject undergoing a bunionectomy, the subject is administered 4.0 mg to 4.5 mg of (E)-2-methoxy-4-((8-methylnon-6-enamido)methyl)phenyl 2-((methylamino)methyl)piperidine-1-carboxylate. In some embodiments of the methods described herein for use in a subject undergoing a bunionectomy, the subject is administered about 5.0 mg of (E)-2-methoxy-4-((8-methylnon-6-enamido)methyl)phenyl 2-((methylamino)methyl)piperidine-1-carboxylate. In some embodiments of the methods described herein for use in a subject undergoing a bunionectomy, the subject is administered about 4.5 mg of (E)-2-methoxy-4-((8-methylnon-6-enamido)methyl)phenyl 2-((methylamino)methyl)piperidine-1-carboxylate. In some embodiments of the methods described herein for use in a subject undergoing a bunionectomy, the subject is administered about 4.2 mg of (E)-2-methoxy-4-((8-methylnon-6-enamido)methyl)phenyl 2-((methylamino)methyl)piperidine-1-carboxylate. In some embodiments of the methods described herein for use in a subject undergoing a bunionectomy, the subject is administered about 4.0 mg of (E)-2-methoxy-4-((8-methylnon-6-enamido)methyl)phenyl 2-((methylamino)methyl)piperidine-1-carboxylate. In some embodiments of the methods described herein for use in a subject undergoing a bunionectomy, the subject is administered about 3.5 mg of (E)-2-methoxy-4-((8-methylnon-6-enamido)methyl)phenyl 2-((methylamino)methyl)piperidine-1-carboxylate. In some embodiments of the methods described herein for use in a subject undergoing a bunionectomy, the subject is administered about 3.0 mg of (E)-2-methoxy-4-((8-methylnon-6-enamido)methyl)phenyl 2-((methylamino)methyl)piperidine-1-carboxylate. In some embodiments of the methods described herein for use in a subject undergoing a bunionectomy, the subject is administered about 2.5 mg of (E)-2-methoxy-4-((8-methylnon-6-enamido)methyl)phenyl 2-((methylamino)methyl)piperidine-1-carboxylate. In some embodiments of the methods described herein for use in a subject undergoing a bunionectomy, the subject is administered about 2.0 mg of (E)-2-methoxy-4-((8-methylnon-6-enamido)methyl)phenyl 2-((methylamino)methyl)piperidine-1-carboxylate. In some embodiments of the methods described herein for use in a subject undergoing a bunionectomy, the subject is administered about 1.5 mg of (E)-2-methoxy-4-((8-methylnon-6-enamido)methyl)phenyl 2-((methylamino)methyl)piperidine-1-carboxylate. In some embodiments of the methods described herein for use in a subject undergoing a bunionectomy, the subject is administered about 1.0 mg of (E)-2-methoxy-4-((8-methylnon-6-enamido)methyl)phenyl 2-((methylamino)methyl)piperidine-1-carboxylate. In some embodiments of the methods described herein for use in a subject undergoing a bunionectomy, the subject is administered about 0.5 mg of (E)-2-methoxy-4-((8-methylnon-6-enamido)methyl)phenyl 2-((methylamino)methyl)piperidine-1-carboxylate.

In some embodiments of the method of treating pain and reducing opioid use in a subject undergoing a bunionectomy described herein, opioid use is reduced by 10% to 90%. In some embodiments of the method of treating pain and reducing opioid use in a subject undergoing a bunionectomy described herein, opioid use is reduced by 10% to 75%. In some embodiments of the method of treating pain and reducing opioid use in a subject undergoing a bunionectomy described herein, opioid use is reduced by 10% to 60%. In some embodiments of the method of treating pain and reducing opioid use in a subject undergoing a bunionectomy described herein, opioid use is reduced by 10% to 50%. In some embodiments of the method of treating pain and reducing opioid use in a subject undergoing a bunionectomy described herein, opioid use is reduced by 20% to 50%. In some embodiments of the method of treating pain and reducing opioid use in a subject undergoing a bunionectomy described herein, opioid use is reduced by 20% to 40%. In some embodiments of the method of treating pain and reducing opioid use in a subject undergoing a bunionectomy described herein, opioid use is reduced by 20% to 30%. In some embodiments of the method of treating pain and reducing opioid use in a subject undergoing a bunionectomy described herein, opioid use is reduced by about 10%. In some embodiments of the method of treating pain and reducing opioid use in a subject undergoing a bunionectomy described herein, opioid use is reduced by about 15%. In some embodiments of the method of treating pain and reducing opioid use in a subject undergoing a bunionectomy described herein, opioid use is reduced by about 20%. In some embodiments of the method of treating pain and reducing opioid use in a subject undergoing a bunionectomy described herein, opioid use is reduced by about 25%. In some embodiments of the method of treating pain and reducing opioid use in a subject undergoing a bunionectomy described herein, opioid use is reduced by about 30%. In some embodiments of the method of treating pain and reducing opioid use in a subject undergoing a bunionectomy described herein, opioid use is reduced by about 35%. In some embodiments of the method of treating pain and reducing opioid use in a subject undergoing a bunionectomy described herein, opioid use is reduced by about 40%. In some embodiments of the method of treating pain and reducing opioid use in a subject undergoing a bunionectomy described herein, opioid use is reduced by about 45%. In some embodiments of the method of treating pain and reducing opioid use in a subject undergoing a bunionectomy described herein, opioid use is reduced by about 50%. In some embodiments of the method of treating pain and reducing opioid use in a subject undergoing a bunionectomy described herein, opioid use is reduced by about 55%. In some embodiments of the method of treating pain and reducing opioid use in a subject undergoing a bunionectomy described herein, opioid use is reduced by about 60%. In some embodiments of the method of treating pain and reducing opioid use in a subject undergoing a bunionectomy described herein, opioid use is reduced by about 65%. In some embodiments of the method of treating pain and reducing opioid use in a subject undergoing a bunionectomy described herein, opioid use is reduced by about 70%. In some embodiments of the method of treating pain and reducing opioid use in a subject undergoing a bunionectomy described herein, opioid use is reduced by about 75%. In some embodiments of the method of treating pain and reducing opioid use in a subject undergoing a bunionectomy described herein, opioid use is reduced by about 80%. In some embodiments of the method of treating pain and reducing opioid use in a subject undergoing a bunionectomy described herein, opioid use is reduced by about 85%. In some embodiments of the method of treating pain and reducing opioid use in a subject undergoing a bunionectomy described herein, opioid use is reduced by about 90%.

In some embodiments, described herein is a method of treating pain in a subject undergoing a unilateral total knee arthroplasty (TKA) comprising administering to the subject 25 mg to 65 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing a unilateral total knee arthroplasty (TKA) comprising administering to the subject 25 mg to 55 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing a unilateral total knee arthroplasty (TKA) comprising administering to the subject 25 mg to 50 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing a unilateral total knee arthroplasty (TKA) comprising administering to the subject 25 mg to 45 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing a unilateral total knee arthroplasty (TKA) comprising administering to the subject 25 mg to 40 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing a unilateral total knee arthroplasty (TKA) comprising administering to the subject 30 mg to 40 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing a unilateral total knee arthroplasty (TKA) comprising administering to the subject 35 mg to 40 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing a unilateral total knee arthroplasty (TKA) comprising administering to the subject about 65 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing a unilateral total knee arthroplasty (TKA) comprising administering to the subject about 60 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing a unilateral total knee arthroplasty (TKA) comprising administering to the subject about 55 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing a unilateral total knee arthroplasty (TKA) comprising administering to the subject about 50 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing a unilateral total knee arthroplasty (TKA) comprising administering to the subject about 45 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing a unilateral total knee arthroplasty (TKA) comprising administering to the subject about 40 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing a unilateral total knee arthroplasty (TKA) comprising administering to the subject about 35 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing a unilateral total knee arthroplasty (TKA) comprising administering to the subject about 30 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing a unilateral total knee arthroplasty (TKA) comprising administering to the subject about 25 mg of (E)-2-methoxy-4-((8-methylnon-6-enamido)methyl)phenyl 2-((methylamino)methyl)piperidine-1-carboxylate (Compound 1).

In some embodiments, described herein is a method of treating pain in a subject undergoing a unilateral total knee arthroplasty (TKA) comprising administering to the subject 25 mg to 65 mg of (E)-2-methoxy-4-((8-methylnon-6-enamido)methyl)phenyl 2-((methylamino)methyl)piperidine-1-carboxylate (Compound 1), wherein the pain is reduced by at least 10% compared to placebo. In some embodiments is a method of treating pain in a subject undergoing a unilateral total knee arthroplasty (TKA) comprising administering to the subject 25 mg to 65 mg of (E)-2-methoxy-4-((8-methylnon-6-enamido)methyl)phenyl 2-((methylamino)methyl)piperidine-1-carboxylate (Compound 1), wherein the pain is reduced by at least 15% compared to placebo. In some embodiments is a method of treating pain in a subject undergoing a unilateral total knee arthroplasty (TKA) comprising administering to the subject 25 mg to 65 mg of (E)-2-methoxy-4-((8-methylnon-6-enamido)methyl)phenyl 2-((methylamino)methyl)piperidine-1-carboxylate (Compound 1), wherein the pain is reduced by at least 20% compared to placebo.

In some embodiments, described herein is a method of treating pain and reducing opioid use in a subject undergoing a unilateral total knee arthroplasty (TKA), comprising administering to the subject 25 mg to 65 of (E)-2-methoxy-4-((8-methylnon-6-enamido)methyl)phenyl 2-((methylamino)methyl)piperidine-1-carboxylate (Compound 1), wherein opioid use is reduced by at least 10%. In some embodiments is a method of treating pain and reducing opioid use in a subject undergoing a unilateral total knee arthroplasty (TKA), comprising administering to the subject 25 mg to 65 of (E)-2-methoxy-4-((8-methylnon-6-enamido)methyl)phenyl 2-((methylamino)methyl)piperidine-1-carboxylate (Compound 1), wherein opioid use is reduced by at least 15%. In some embodiments is a method of treating pain and reducing opioid use in a subject undergoing a unilateral total knee arthroplasty (TKA), comprising administering to the subject 25 mg to 65 of (E)-2-methoxy-4-((8-methylnon-6-enamido)methyl)phenyl 2-((methylamino)methyl)piperidine-1-carboxylate (Compound 1), wherein opioid use is reduced by at least 20%. In some embodiments is a method of treating pain and reducing opioid use in a subject undergoing a unilateral total knee arthroplasty (TKA), comprising administering to the subject 25 mg to 65 of (E)-2-methoxy-4-((8-methylnon-6-enamido)methyl)phenyl 2-((methylamino)methyl)piperidine-1-carboxylate (Compound 1), wherein opioid use is reduced by at least 25%. In some embodiments is a method of treating pain and reducing opioid use in a subject undergoing a unilateral total knee arthroplasty (TKA), comprising administering to the subject 25 mg to 65 of (E)-2-methoxy-4-((8-methylnon-6-enamido)methyl)phenyl 2-((methylamino)methyl)piperidine-1-carboxylate (Compound 1), wherein opioid use is reduced by at least 30%.

In some embodiments of the methods described herein for use in a subject undergoing a unilateral total knee arthroplasty (TKA), the subject is administered 25 mg to 55 mg of (E)-2-methoxy-4-((8-methylnon-6-enamido)methyl)phenyl 2-((methylamino)methyl)piperidine-1-carboxylate. In some embodiments of the methods described herein for use in a subject undergoing a unilateral total knee arthroplasty (TKA), the subject is administered 25 mg to 50 mg of (E)-2-methoxy-4-((8-methylnon-6-enamido)methyl)phenyl 2-((methylamino)methyl)piperidine-1-carboxylate. In some embodiments of the methods described herein for use in a subject undergoing a unilateral total knee arthroplasty (TKA), the subject is administered 25 mg to 45 mg of (E)-2-methoxy-4-((8-methylnon-6-enamido)methyl)phenyl 2-((methylamino)methyl)piperidine-1-carboxylate. In some embodiments of the methods described herein for use in a subject undergoing a unilateral total knee arthroplasty (TKA), the subject is administered 25 mg to 40 mg of (E)-2-methoxy-4-((8-methylnon-6-enamido)methyl)phenyl 2-((methylamino)methyl)piperidine-1-carboxylate. In some embodiments of the methods described herein for use in a subject undergoing a unilateral total knee arthroplasty (TKA), the subject is administered 30 mg to 40 mg of (E)-2-methoxy-4-((8-methylnon-6-enamido)methyl)phenyl 2-((methylamino)methyl)piperidine-1-carboxylate. In some embodiments of the methods described herein for use in a subject undergoing a unilateral total knee arthroplasty (TKA), the subject is administered 35 mg to 40 mg of (E)-2-methoxy-4-((8-methylnon-6-enamido)methyl)phenyl 2-((methylamino)methyl)piperidine-1-carboxylate. In some embodiments of the methods described herein for use in a subject undergoing a unilateral total knee arthroplasty (TKA), the subject is administered about 60 mg of (E)-2-methoxy-4-((8-methylnon-6-enamido)methyl)phenyl 2-((methylamino)methyl)piperidine-1-carboxylate. In some embodiments of the methods described herein for use in a subject undergoing a unilateral total knee arthroplasty (TKA), the subject is administered about 55 mg of (E)-2-methoxy-4-((8-methylnon-6-enamido)methyl)phenyl 2-((methylamino)methyl)piperidine-1-carboxylate. In some embodiments of the methods described herein for use in a subject undergoing a unilateral total knee arthroplasty (TKA), the subject is administered about 50 mg of (E)-2-methoxy-4-((8-methylnon-6-enamido)methyl)phenyl 2-((methylamino)methyl)piperidine-1-carboxylate. In some embodiments of the methods described herein for use in a subject undergoing a unilateral total knee arthroplasty (TKA), the subject is administered about 45 mg of (E)-2-methoxy-4-((8-methylnon-6-enamido)methyl)phenyl 2-((methylamino)methyl)piperidine-1-carboxylate. In some embodiments of the methods described herein for use in a subject undergoing a unilateral total knee arthroplasty (TKA), the subject is administered about 40 mg of (E)-2-methoxy-4-((8-methylnon-6-enamido)methyl)phenyl 2-((methylamino)methyl)piperidine-1-carboxylate. In some embodiments of the methods described herein for use in a subject undergoing a unilateral total knee arthroplasty (TKA), the subject is administered about 35 mg of (E)-2-methoxy-4-((8-methylnon-6-enamido)methyl)phenyl 2-((methylamino)methyl)piperidine-1-carboxylate. In some embodiments of the methods described herein for use in a subject undergoing a unilateral total knee arthroplasty (TKA), the subject is administered about 30 mg of (E)-2-methoxy-4-((8-methylnon-6-enamido)methyl)phenyl 2-((methylamino)methyl)piperidine-1-carboxylate. In some embodiments of the methods described herein for use in a subject undergoing a unilateral total knee arthroplasty (TKA), the subject is administered about 25 mg of (E)-2-methoxy-4-((8-methylnon-6-enamido)methyl)phenyl 2-((methylamino)methyl)piperidine-1-carboxylate.

In some embodiments of the method of treating pain and reducing opioid use in a subject undergoing a unilateral total knee arthroplasty (TKA) described herein, opioid use is reduced by 10% to 90%. In some embodiments of the method of treating pain and reducing opioid use in a subject undergoing a unilateral total knee arthroplasty (TKA) described herein, opioid use is reduced by 10% to 75%. In some embodiments of the method of treating pain and reducing opioid use in a subject undergoing a unilateral total knee arthroplasty (TKA) described herein, opioid use is reduced by 10% to 60%. In some embodiments of the method of treating pain and reducing opioid use in a subject undergoing a unilateral total knee arthroplasty (TKA) described herein, opioid use is reduced by 10% to 50%. In some embodiments of the method of treating pain and reducing opioid use in a subject undergoing a unilateral total knee arthroplasty (TKA) described herein, opioid use is reduced by 20% to 50%. In some embodiments of the method of treating pain and reducing opioid use in a subject undergoing a unilateral total knee arthroplasty (TKA) described herein, opioid use is reduced by 20% to 40%. In some embodiments of the method of treating pain and reducing opioid use in a subject undergoing a unilateral total knee arthroplasty (TKA) described herein, opioid use is reduced by 20% to 30%. In some embodiments of the method of treating pain and reducing opioid use in a subject undergoing a unilateral total knee arthroplasty (TKA) described herein, opioid use is reduced by about 10%. In some embodiments of the method of treating pain and reducing opioid use in a subject undergoing a unilateral total knee arthroplasty (TKA) described herein, opioid use is reduced by about 15%. In some embodiments of the method of treating pain and reducing opioid use in a subject undergoing a unilateral total knee arthroplasty (TKA) described herein, opioid use is reduced by about 20%. In some embodiments of the method of treating pain and reducing opioid use in a subject undergoing a unilateral total knee arthroplasty (TKA) described herein, opioid use is reduced by about 25%. In some embodiments of the method of treating pain and reducing opioid use in a subject undergoing a unilateral total knee arthroplasty (TKA) described herein, opioid use is reduced by about 30%. In some embodiments of the method of treating pain and reducing opioid use in a subject undergoing a unilateral total knee arthroplasty (TKA) described herein, opioid use is reduced by about 35%. In some embodiments of the method of treating pain and reducing opioid use in a subject undergoing a unilateral total knee arthroplasty (TKA) described herein, opioid use is reduced by about 40%. In some embodiments of the method of treating pain and reducing opioid use in a subject undergoing a unilateral total knee arthroplasty (TKA) described herein, opioid use is reduced by about 45%. In some embodiments of the method of treating pain and reducing opioid use in a subject undergoing a unilateral total knee arthroplasty (TKA) described herein, opioid use is reduced by about 50%. In some embodiments of the method of treating pain and reducing opioid use in a subject undergoing a unilateral total knee arthroplasty (TKA) described herein, opioid use is reduced by about 55%. In some embodiments of the method of treating pain and reducing opioid use in a subject undergoing a unilateral total knee arthroplasty (TKA) described herein, opioid use is reduced by about 60%. In some embodiments of the method of treating pain and reducing opioid use in a subject undergoing a unilateral total knee arthroplasty (TKA) described herein, opioid use is reduced by about 65%. In some embodiments of the method of treating pain and reducing opioid use in a subject undergoing a unilateral total knee arthroplasty (TKA) described herein, opioid use is reduced by about 70%. In some embodiments of the method of treating pain and reducing opioid use in a subject undergoing a unilateral total knee arthroplasty (TKA) described herein, opioid use is reduced by about 75%. In some embodiments of the method of treating pain and reducing opioid use in a subject undergoing a unilateral total knee arthroplasty (TKA) described herein, opioid use is reduced by about 80%. In some embodiments of the method of treating pain and reducing opioid use in a subject undergoing a unilateral total knee arthroplasty (TKA) described herein, opioid use is reduced by about 85%. In some embodiments of the method of treating pain and reducing opioid use in a subject undergoing a unilateral total knee arthroplasty (TKA) described herein, opioid use is reduced by about 90%.

In some embodiments, described herein is a method of treating pain in a subject undergoing laparotomy comprising administering to the subject 25 mg to 65 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy comprising administering to the subject 25 mg to 55 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy comprising administering to the subject 25 mg to 50 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy comprising administering to the subject 25 mg to 45 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy comprising administering to the subject 25 mg to 40 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy comprising administering to the subject 30 mg to 40 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy comprising administering to the subject 35 mg to 40 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy comprising administering to the subject about 65 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy comprising administering to the subject about 60 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy comprising administering to the subject about 55 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy comprising administering to the subject about 50 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy comprising administering to the subject about 45 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy comprising administering to the subject about 40 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy comprising administering to the subject about 35 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy comprising administering to the subject about 30 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy comprising administering to the subject about 25 mg of (E)-2-methoxy-4-((8-methylnon-6-enamido)methyl)phenyl 2-((methylamino)methyl)piperidine-1-carboxylate (Compound 1).

In some embodiments, described herein is a method of treating pain in a subject undergoing laparotomy comprising administering to the subject 25 mg to 65 mg of (E)-2-methoxy-4-((8-methylnon-6-enamido)methyl)phenyl 2-((methylamino)methyl)piperidine-1-carboxylate (Compound 1), wherein the pain is reduced by at least 10% compared to placebo. In some embodiments is a method of treating pain in a subject undergoing laparotomy comprising administering to the subject 25 mg to 65 mg of (E)-2-methoxy-4-((8-methylnon-6-enamido)methyl)phenyl 2-((methylamino)methyl)piperidine-1-carboxylate (Compound 1), wherein the pain is reduced by at least 15% compared to placebo. In some embodiments is a method of treating pain in a subject undergoing laparotomy comprising administering to the subject 25 mg to 65 mg of (E)-2-methoxy-4-((8-methylnon-6-enamido)methyl)phenyl 2-((methylamino)methyl)piperidine-1-carboxylate (Compound 1), wherein the pain is reduced by at least 20% compared to placebo.

In some embodiments, described herein is a method of treating pain and reducing opioid use in a subject undergoing laparotomy, comprising administering to the subject 25 mg to 65 of (E)-2-methoxy-4-((8-methylnon-6-enamido)methyl)phenyl 2-((methylamino)methyl)piperidine-1-carboxylate (Compound 1), wherein opioid use is reduced by at least 10%. In some embodiments is a method of treating pain and reducing opioid use in a subject undergoing laparotomy, comprising administering to the subject 25 mg to 65 of (E)-2-methoxy-4-((8-methylnon-6-enamido)methyl)phenyl 2-((methylamino)methyl)piperidine-1-carboxylate (Compound 1), wherein opioid use is reduced by at least 15%. In some embodiments is a method of treating pain and reducing opioid use in a subject undergoing laparotomy, comprising administering to the subject 25 mg to 65 of (E)-2-methoxy-4-((8-methylnon-6-enamido)methyl)phenyl 2-((methylamino)methyl)piperidine-1-carboxylate (Compound 1), wherein opioid use is reduced by at least 20%. In some embodiments is a method of treating pain and reducing opioid use in a subject undergoing laparotomy, comprising administering to the subject 25 mg to 65 of (E)-2-methoxy-4-((8-methylnon-6-enamido)methyl)phenyl 2-((methylamino)methyl)piperidine-1-carboxylate (Compound 1), wherein opioid use is reduced by at least 25%. In some embodiments is a method of treating pain and reducing opioid use in a subject undergoing laparotomy, comprising administering to the subject 25 mg to 65 of (E)-2-methoxy-4-((8-methylnon-6-enamido)methyl)phenyl 2-((methylamino)methyl)piperidine-1-carboxylate (Compound 1), wherein opioid use is reduced by at least 30%.

In some embodiments of the methods described herein for use in a subject undergoing laparotomy, the subject is administered 25 mg to 55 mg of (E)-2-methoxy-4-((8-methylnon-6-enamido)methyl)phenyl 2-((methylamino)methyl)piperidine-1-carboxylate. In some embodiments of the methods described herein for use in a subject undergoing laparotomy, the subject is administered 25 mg to 50 mg of (E)-2-methoxy-4-((8-methylnon-6-enamido)methyl)phenyl 2-((methylamino)methyl)piperidine-1-carboxylate. In some embodiments of the methods described herein for use in a subject undergoing laparotomy, the subject is administered 25 mg to 45 mg of (E)-2-methoxy-4-((8-methylnon-6-enamido)methyl)phenyl 2-((methylamino)methyl)piperidine-1-carboxylate. In some embodiments of the methods described herein for use in a subject undergoing laparotomy, the subject is administered 25 mg to 40 mg of (E)-2-methoxy-4-((8-methylnon-6-enamido)methyl)phenyl 2-((methylamino)methyl)piperidine-1-carboxylate. In some embodiments of the methods described herein for use in a subject undergoing laparotomy, the subject is administered 30 mg to 40 mg of (E)-2-methoxy-4-((8-methylnon-6-enamido)methyl)phenyl 2-((methylamino)methyl)piperidine-1-carboxylate. In some embodiments of the methods described herein for use in a subject undergoing laparotomy, the subject is administered 35 mg to 40 mg of (E)-2-methoxy-4-((8-methylnon-6-enamido)methyl)phenyl 2-((methylamino)methyl)piperidine-1-carboxylate. In some embodiments of the methods described herein for use in a subject undergoing laparotomy, the subject is administered about 60 mg of (E)-2-methoxy-4-((8-methylnon-6-enamido)methyl)phenyl 2-((methylamino)methyl)piperidine-1-carboxylate. In some embodiments of the methods described herein for use in a subject undergoing laparotomy, the subject is administered about 55 mg of (E)-2-methoxy-4-((8-methylnon-6-enamido)methyl)phenyl 2-((methylamino)methyl)piperidine-1-carboxylate. In some embodiments of the methods described herein for use in a subject undergoing laparotomy, the subject is administered about 50 mg of (E)-2-methoxy-4-((8-methylnon-6-enamido)methyl)phenyl 2-((methylamino)methyl)piperidine-1-carboxylate. In some embodiments of the methods described herein for use in a subject undergoing laparotomy, the subject is administered about 45 mg of (E)-2-methoxy-4-((8-methylnon-6-enamido)methyl)phenyl 2-((methylamino)methyl)piperidine-1-carboxylate. In some embodiments of the methods described herein for use in a subject undergoing laparotomy, the subject is administered about 40 mg of (E)-2-methoxy-4-((8-methylnon-6-enamido)methyl)phenyl 2-((methylamino)methyl)piperidine-1-carboxylate. In some embodiments of the methods described herein for use in a subject undergoing laparotomy, the subject is administered about 35 mg of (E)-2-methoxy-4-((8-methylnon-6-enamido)methyl)phenyl 2-((methylamino)methyl)piperidine-1-carboxylate. In some embodiments of the methods described herein for use in a subject undergoing laparotomy, the subject is administered about 30 mg of (E)-2-methoxy-4-((8-methylnon-6-enamido)methyl)phenyl 2-((methylamino)methyl)piperidine-1-carboxylate. In some embodiments of the methods described herein for use in a subject undergoing laparotomy, the subject is administered about 25 mg of (E)-2-methoxy-4-((8-methylnon-6-enamido)methyl)phenyl 2-((methylamino)methyl)piperidine-1-carboxylate.

In some embodiments of the method of treating pain and reducing opioid use in a subject undergoing laparotomy described herein, opioid use is reduced by 10% to 90%. In some embodiments of the method of treating pain and reducing opioid use in a subject undergoing laparotomy described herein, opioid use is reduced by 10% to 75%. In some embodiments of the method of treating pain and reducing opioid use in a subject undergoing laparotomy described herein, opioid use is reduced by 10% to 60%. In some embodiments of the method of treating pain and reducing opioid use in a subject undergoing laparotomy described herein, opioid use is reduced by 10% to 50%. In some embodiments of the method of treating pain and reducing opioid use in a subject undergoing laparotomy described herein, opioid use is reduced by 20% to 50%. In some embodiments of the method of treating pain and reducing opioid use in a subject undergoing laparotomy described herein, opioid use is reduced by 20% to 40%. In some embodiments of the method of treating pain and reducing opioid use in a subject undergoing laparotomy described herein, opioid use is reduced by 20% to 30%. In some embodiments of the method of treating pain and reducing opioid use in a subject undergoing laparotomy described herein, opioid use is reduced by about 10%. In some embodiments of the method of treating pain and reducing opioid use in a subject undergoing laparotomy described herein, opioid use is reduced by about 15%. In some embodiments of the method of treating pain and reducing opioid use in a subject undergoing laparotomy described herein, opioid use is reduced by about 20%. In some embodiments of the method of treating pain and reducing opioid use in a subject undergoing laparotomy described herein, opioid use is reduced by about 25%. In some embodiments of the method of treating pain and reducing opioid use in a subject undergoing laparotomy described herein, opioid use is reduced by about 30%. In some embodiments of the method of treating pain and reducing opioid use in a subject undergoing laparotomy described herein, opioid use is reduced by about 35%. In some embodiments of the method of treating pain and reducing opioid use in a subject undergoing laparotomy described herein, opioid use is reduced by about 40%. In some embodiments of the method of treating pain and reducing opioid use in a subject undergoing laparotomy described herein, opioid use is reduced by about 45%. In some embodiments of the method of treating pain and reducing opioid use in a subject undergoing laparotomy described herein, opioid use is reduced by about 50%. In some embodiments of the method of treating pain and reducing opioid use in a subject undergoing laparotomy described herein, opioid use is reduced by about 55%. In some embodiments of the method of treating pain and reducing opioid use in a subject undergoing laparotomy described herein, opioid use is reduced by about 60%. In some embodiments of the method of treating pain and reducing opioid use in a subject undergoing laparotomy described herein, opioid use is reduced by about 65%. In some embodiments of the method of treating pain and reducing opioid use in a subject undergoing laparotomy described herein, opioid use is reduced by about 70%. In some embodiments of the method of treating pain and reducing opioid use in a subject undergoing laparotomy described herein, opioid use is reduced by about 75%. In some embodiments of the method of treating pain and reducing opioid use in a subject undergoing laparotomy described herein, opioid use is reduced by about 80%. In some embodiments of the method of treating pain and reducing opioid use in a subject undergoing laparotomy described herein, opioid use is reduced by about 85%. In some embodiments of the method of treating pain and reducing opioid use in a subject undergoing laparotomy described herein, opioid use is reduced by about 90%.

In some embodiments of the methods described herein, the laparotomy is selected from a laparotomy to repair a ventral hernia, a C-section, hysterectomy, intestinal resection, and nephrectomy.

In some embodiments, described herein is a method of treating pain in a subject undergoing laparotomy to repair a ventral hernia comprising administering to the subject 25 mg to 65 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy to repair a ventral hernia comprising administering to the subject 25 mg to 55 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy to repair a ventral hernia comprising administering to the subject 25 mg to 50 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy to repair a ventral hernia comprising administering to the subject 25 mg to 45 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy to repair a ventral hernia comprising administering to the subject 25 mg to 40 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy to repair a ventral hernia comprising administering to the subject 30 mg to 40 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy to repair a ventral hernia comprising administering to the subject 35 mg to 40 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy to repair a ventral hernia comprising administering to the subject about 65 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy to repair a ventral hernia comprising administering to the subject about 60 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy to repair a ventral hernia comprising administering to the subject about 55 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy to repair a ventral hernia comprising administering to the subject about 50 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy to repair a ventral hernia comprising administering to the subject about 45 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy to repair a ventral hernia comprising administering to the subject about 40 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy to repair a ventral hernia comprising administering to the subject about 35 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy to repair a ventral hernia comprising administering to the subject about 30 mg of (E)-2-methoxy-4-((8-methylnon-6-enamido)methyl)phenyl 2-((methylamino)methyl)piperidine-1-carboxylate (Compound 1). In some embodiments, described herein is a method of treating pain in a subject undergoing laparotomy to repair a ventral hernia comprising administering to the subject about 25 mg of (E)-2-methoxy-4-((8-methylnon-6-enamido)methyl)phenyl 2-((methylamino)methyl)piperidine-1-carboxylate (Compound 1).

In some embodiments, described herein is a method of treating pain in a subject undergoing laparotomy to repair a ventral hernia comprising administering to the subject 25 mg to 65 mg of (E)-2-methoxy-4-((8-methylnon-6-enamido)methyl)phenyl 2-((methylamino)methyl)piperidine-1-carboxylate (Compound 1), wherein the pain is reduced by at least 10% compared to placebo. In some embodiments is a method of treating pain in a subject undergoing laparotomy to repair a ventral hernia comprising administering to the subject 25 mg to 65 mg of (E)-2-methoxy-4-((8-methylnon-6-enamido)methyl)phenyl 2-((methylamino)methyl)piperidine-1-carboxylate (Compound 1), wherein the pain is reduced by at least 15% compared to placebo. In some embodiments is a method of treating pain in a subject undergoing laparotomy to repair a ventral hernia comprising administering to the subject 25 mg to 65 mg of (E)-2-methoxy-4-((8-methylnon-6-enamido)methyl)phenyl 2-((methylamino)methyl)piperidine-1-carboxylate (Compound 1), wherein the pain is reduced by at least 20% compared to placebo.

In some embodiments, described herein is a method of treating pain and reducing opioid use in a subject undergoing laparotomy to repair a ventral hernia, comprising administering to the subject 25 mg to 65 of (E)-2-methoxy-4-((8-methylnon-6-enamido)methyl)phenyl 2-((methylamino)methyl)piperidine-1-carboxylate (Compound 1), wherein opioid use is reduced by at least 10%. In some embodiments is a method of treating pain and reducing opioid use in a subject undergoing laparotomy to repair a ventral hernia, comprising administering to the subject 25 mg to 65 of (E)-2-methoxy-4-((8-methylnon-6-enamido)methyl)phenyl 2-((methylamino)methyl)piperidine-1-carboxylate (Compound 1), wherein opioid use is reduced by at least 15%. In some embodiments is a method of treating pain and reducing opioid use in a subject undergoing laparotomy to repair a ventral hernia, comprising administering to the subject 25 mg to 65 of (E)-2-methoxy-4-((8-methylnon-6-enamido)methyl)phenyl 2-((methylamino)methyl)piperidine-1-carboxylate (Compound 1), wherein opioid use is reduced by at least 20%. In some embodiments is a method of treating pain and reducing opioid use in a subject undergoing laparotomy to repair a ventral hernia, comprising administering to the subject 25 mg to 65 of (E)-2-methoxy-4-((8-methylnon-6-enamido)methyl)phenyl 2-((methylamino)methyl)piperidine-1-carboxylate (Compound 1), wherein opioid use is reduced by at least 25%. In some embodiments is a method of treating pain and reducing opioid use in a subject undergoing laparotomy to repair a ventral hernia, comprising administering to the subject 25 mg to 65 of (E)-2-methoxy-4-((8-methylnon-6-enamido)methyl)phenyl 2-((methylamino)methyl)piperidine-1-carboxylate (Compound 1), wherein opioid use is reduced by at least 30%.

In some embodiments of the methods described herein for use in a subject undergoing laparotomy to repair a ventral hernia, the subject is administered 25 mg to 55 mg of (E)-2-methoxy-4-((8-methylnon-6-enamido)methyl)phenyl 2-((methylamino)methyl)piperidine-1-carboxylate. In some embodiments of the methods described herein for use in a subject undergoing laparotomy to repair a ventral hernia, the subject is administered 25 mg to 50 mg of (E)-2-methoxy-4-((8-methylnon-6-enamido)methyl)phenyl 2-((methylamino)methyl)piperidine-1-carboxylate. In some embodiments of the methods described herein for use in a subject undergoing laparotomy to repair a ventral hernia, the subject is administered 25 mg to 45 mg of (E)-2-methoxy-4-((8-methylnon-6-enamido)methyl)phenyl 2-((methylamino)methyl)piperidine-1-carboxylate. In some embodiments of the methods described herein for use in a subject undergoing laparotomy to repair a ventral hernia, the subject is administered 25 mg to 40 mg of (E)-2-methoxy-4-((8-methylnon-6-enamido)methyl)phenyl 2-((methylamino)methyl)piperidine-1-carboxylate. In some embodiments of the methods described herein for use in a subject undergoing laparotomy to repair a ventral hernia, the subject is administered 30 mg to 40 mg of (E)-2-methoxy-4-((8-methylnon-6-enamido)methyl)phenyl 2-((methylamino)methyl)piperidine-1-carboxylate. In some embodiments of the methods described herein for use in a subject undergoing laparotomy to repair a ventral hernia, the subject is administered 35 mg to 40 mg of (E)-2-methoxy-4-((8-methylnon-6-enamido)methyl)phenyl 2-((methylamino)methyl)piperidine-1-carboxylate. In some embodiments of the methods described herein for use in a subject undergoing laparotomy to repair a ventral hernia, the subject is administered about 60 mg of (E)-2-methoxy-4-((8-methylnon-6-enamido)methyl)phenyl 2-((methylamino)methyl)piperidine-1-carboxylate. In some embodiments of the methods described herein for use in a subject undergoing laparotomy to repair a ventral hernia, the subject is administered about 55 mg of (E)-2-methoxy-4-((8-methylnon-6-enamido)methyl)phenyl 2-((methylamino)methyl)piperidine-1-carboxylate. In some embodiments of the methods described herein for use in a subject undergoing laparotomy to repair a ventral hernia, the subject is administered about 50 mg of (E)- 2-methoxy-4-((8-methylnon-6-enamido)methyl)phenyl 2-((methylamino)methyl)piperidine-1-carboxylate. In some embodiments of the methods described herein for use in a subject undergoing laparotomy to repair a ventral hernia, the subject is administered about 45 mg of (E)-2-methoxy-4-((8-methylnon-6-enamido)methyl)phenyl 2-((methylamino)methyl)piperidine-1-carboxylate. In some embodiments of the methods described herein for use in a subject undergoing laparotomy to repair a ventral hernia, the subject is administered about 40 mg of (E)-2-methoxy-4-((8-methylnon-6-enamido)methyl)phenyl 2-((methylamino)methyl)piperidine-1-carboxylate. In some embodiments of the methods described herein for use in a subject undergoing laparotomy to repair a ventral hernia, the subject is administered about 35 mg of (E)-2-methoxy-4-((8-methylnon-6-enamido)methyl)phenyl 2-((methylamino)methyl)piperidine-1-carboxylate. In some embodiments of the methods described herein for use in a subject undergoing laparotomy to repair a ventral hernia, the subject is administered about 30 mg of (E)-2-methoxy-4-((8-methylnon-6-enamido)methyl)phenyl 2-((methylamino)methyl)piperidine-1-carboxylate. In some embodiments of the methods described herein for use in a subject undergoing laparotomy to repair a ventral hernia, the subject is administered about 25 mg of (E)-2-methoxy-4-((8-methylnon-6-enamido)methyl)phenyl 2-((methylamino)methyl)piperidine-1-carboxylate.

In some embodiments of the method of treating pain and reducing opioid use in a subject undergoing laparotomy to repair a ventral hernia described herein, opioid use is reduced by 10% to 90%. In some embodiments of the method of treating pain and reducing opioid use in a subject undergoing laparotomy to repair a ventral hernia described herein, opioid use is reduced by 10% to 75%. In some embodiments of the method of treating pain and reducing opioid use in a subject undergoing laparotomy to repair a ventral hernia described herein, opioid use is reduced by 10% to 60%. In some embodiments of the method of treating pain and reducing opioid use in a subject undergoing laparotomy to repair a ventral hernia described herein, opioid use is reduced by 10% to 50%. In some embodiments of the method of treating pain and reducing opioid use in a subject undergoing laparotomy to repair a ventral hernia described herein, opioid use is reduced by 20% to 50%. In some embodiments of the method of treating pain and reducing opioid use in a subject undergoing laparotomy to repair a ventral hernia described herein, opioid use is reduced by 20% to 40%. In some embodiments of the method of treating pain and reducing opioid use in a subject undergoing laparotomy to repair a ventral hernia described herein, opioid use is reduced by 20% to 30%. In some embodiments of the method of treating pain and reducing opioid use in a subject undergoing laparotomy to repair a ventral hernia described herein, opioid use is reduced by about 10%. In some embodiments of the method of treating pain and reducing opioid use in a subject undergoing laparotomy to repair a ventral hernia described herein, opioid use is reduced by about 15%. In some embodiments of the method of treating pain and reducing opioid use in a subject undergoing laparotomy to repair a ventral hernia described herein, opioid use is reduced by about 20%. In some embodiments of the method of treating pain and reducing opioid use in a subject undergoing laparotomy to repair a ventral hernia described herein, opioid use is reduced by about 25%. In some embodiments of the method of treating pain and reducing opioid use in a subject undergoing laparotomy to repair a ventral hernia described herein, opioid use is reduced by about 30%. In some embodiments of the method of treating pain and reducing opioid use in a subject undergoing laparotomy to repair a ventral hernia described herein, opioid use is reduced by about 35%. In some embodiments of the method of treating pain and reducing opioid use in a subject undergoing laparotomy to repair a ventral hernia described herein, opioid use is reduced by about 40%. In some embodiments of the method of treating pain and reducing opioid use in a subject undergoing laparotomy to repair a ventral hernia described herein, opioid use is reduced by about 45%. In some embodiments of the method of treating pain and reducing opioid use in a subject undergoing laparotomy to repair a ventral hernia described herein, opioid use is reduced by about 50%. In some embodiments of the method of treating pain and reducing opioid use in a subject undergoing laparotomy to repair a ventral hernia described herein, opioid use is reduced by about 55%. In some embodiments of the method of treating pain and reducing opioid use in a subject undergoing laparotomy to repair a ventral hernia described herein, opioid use is reduced by about 60%. In some embodiments of the method of treating pain and reducing opioid use in a subject undergoing laparotomy to repair a ventral hernia described herein, opioid use is reduced by about 65%. In some embodiments of the method of treating pain and reducing opioid use in a subject undergoing laparotomy to repair a ventral hernia described herein, opioid use is reduced by about 70%. In some embodiments of the method of treating pain and reducing opioid use in a subject undergoing laparotomy to repair a ventral hernia described herein, opioid use is reduced by about 75%. In some embodiments of the method of treating pain and reducing opioid use in a subject undergoing laparotomy to repair a ventral hernia described herein, opioid use is reduced by about 80%. In some embodiments of the method of treating pain and reducing opioid use in a subject undergoing laparotomy to repair a ventral hernia described herein, opioid use is reduced by about 85%. In some embodiments of the method of treating pain and reducing opioid use in a subject undergoing laparotomy to repair a ventral hernia described herein, opioid use is reduced by about 90%.

In some embodiments of the methods of treating pain in a subject described herein, the subject is undergoing an abdominal incision. In some embodiments of the methods of treating pain in a subject described herein, the subject is undergoing an abdominoplasty. In some embodiments of the methods of treating pain in a subject described herein, the subject is undergoing repair of an inguinal hernia. In some embodiments of the methods of treating pain in a subject described herein, the subject is undergoing a laparotomy. In some embodiments of the methods of treating pain in a subject described herein, the subject is undergoing a laparotomy selected from a laparotomy to repair a ventral hernia, a C-section, hysterectomy, intestinal resection, and nephrectomy. In some embodiments of the methods of treating pain in a subject described herein, the subject is undergoing a laparotomy to repair a ventral hernia. In some embodiments of the methods of treating pain in a subject described herein, the subject is undergoing a C-section. In some embodiments of the methods of treating pain in a subject described herein, the subject is undergoing a hysterectomy. In some embodiments of the methods of treating pain in a subject described herein, the subject is undergoing a nephrectomy. In some embodiments of the methods of treating pain in a subject described herein, the subject is undergoing general surgery. In some embodiments of the methods of treating pain in a subject described herein, the subject is undergoing obstetric and gynecological surgery. In some embodiments of the methods of treating pain in a subject described herein, the subject is undergoing plastic surgery.

In some embodiments of the methods described herein, (E)-2-methoxy-4-((8-methylnon-6-enamido)methyl)phenyl 2-((methylamino)methyl)piperidine-1-carboxylate (Compound 1) is administered by infiltration and instillation. In some embodiments of the methods described herein, 10% to 35% of (E)-2-methoxy-4-((8-methylnon-6-enamido)methyl)phenyl 2-((methylamino)methyl)piperidine-1-carboxylate (Compound 1) is administered by instillation and 65% to 90% of (E)-2-methoxy-4-((8-methylnon-6-enamido)methyl)phenyl 2-((methylamino)methyl)piperidine-1-carboxylate (Compound 1) is administered by infiltration. In some embodiments of the methods described herein, 15% to 35% of (E)-2-methoxy-4-((8-methylnon-6-enamido)methyl)phenyl 2-((methylamino)methyl)piperidine-1-carboxylate (Compound 1) is administered by instillation and 65% to 85% of (E)-2-methoxy-4-((8-methylnon-6-enamido)methyl)phenyl 2-((methylamino)methyl)piperidine-1-carboxylate (Compound 1) is administered by infiltration. In some embodiments of the methods described herein, 15% to 30% of (E)-2-methoxy-4-((8-methylnon-6-enamido)methyl)phenyl 2-((methylamino)methyl)piperidine-1-carboxylate (Compound 1) is administered by instillation and 70% to 85% of (E)-2-methoxy-4-((8-methylnon-6-enamido)methyl)phenyl 2-((methylamino)methyl)piperidine-1-carboxylate (Compound 1) is administered by infiltration. In some embodiments of the methods described herein, 20% to 30% of (E)-2-methoxy-4-((8-methylnon-6-enamido)methyl)phenyl 2-((methylamino)methyl)piperidine-1-carboxylate (Compound 1) is administered by instillation and 70% to 80% of (E)-2-methoxy-4-((8-methylnon-6-enamido)methyl)phenyl 2-((methylamino)methyl)piperidine-1-carboxylate (Compound 1) is administered by infiltration. In some embodiments of the methods described herein, 20% to 25% of (E)-2-methoxy-4-((8-methylnon-6-enamido)methyl)phenyl 2-((methylamino)methyl)piperidine-1-carboxylate (Compound 1) is administered by instillation and 75% to 80% of (E)-2-methoxy-4-((8-methylnon-6-enamido)methyl)phenyl 2-((methylamino)methyl)piperidine-1-carboxylate (Compound 1) is administered by infiltration. In some embodiments of the methods described herein, about 10% of (E)-2-methoxy-4-((8-methylnon-6-enamido)methyl)phenyl 2-((methylamino)methyl)piperidine-1-carboxylate (Compound 1) is administered by instillation and 90% of (E)-2-methoxy-4-((8-methylnon-6-enamido)methyl)phenyl 2-((methylamino)methyl)piperidine-1-carboxylate (Compound 1) is administered by infiltration. In some embodiments of the methods described herein, about 15% of (E)-2-methoxy-4-((8-methylnon-6-enamido)methyl)phenyl 2-((methylamino)methyl)piperidine-1-carboxylate (Compound 1) is administered by instillation and 85% of (E)-2-methoxy-4-((8-methylnon-6-enamido)methyl)phenyl 2-((methylamino)methyl)piperidine-1-carboxylate (Compound 1) is administered by infiltration. In some embodiments of the methods described herein, about 20% of (E)-2-methoxy-4-((8-methylnon-6-enamido)methyl)phenyl 2-((methylamino)methyl)piperidine-1-carboxylate (Compound 1) is administered by instillation and 80% of (E)-2-methoxy-4-((8-methylnon-6-enamido)methyl)phenyl 2-((methylamino)methyl)piperidine-1-carboxylate (Compound 1) is administered by infiltration. In some embodiments of the methods described herein, about 25% of (E)-2-methoxy-4-((8-methylnon-6-enamido)methyl)phenyl 2-((methylamino)methyl)piperidine-1-carboxylate (Compound 1) is administered by instillation and 75% of (E)-2-methoxy-4-((8-methylnon-6-enamido)methyl)phenyl 2-((methylamino)methyl)piperidine-1-carboxylate (Compound 1) is administered by infiltration. In some embodiments of the methods described herein, about 30% of (E)-2-methoxy-4-((8-methylnon-6-enamido)methyl)phenyl 2-((methylamino)methyl)piperidine-1-carboxylate (Compound 1) is administered by instillation and 70% of (E)-2-methoxy-4-((8-methylnon-6-enamido)methyl)phenyl 2-((methylamino)methyl)piperidine-1-carboxylate (Compound 1) is administered by infiltration. In some embodiments of the methods described herein, about 35% of (E)-2-methoxy-4-((8-methylnon-6-enamido)methyl)phenyl 2-((methylamino)methyl)piperidine-1-carboxylate (Compound 1) is administered by instillation and 65% of (E)-2-methoxy-4-((8-methylnon-6-enamido)methyl)phenyl 2-((methylamino)methyl)piperidine-1-carboxylate (Compound 1) is administered by infiltration.

### EXAMPLES

These examples are provided for illustrative purposes only and not to limit the scope of the claims provided herein.

### Example 1: A Phase 2, Randomized, Double-blind, Placebo-controlled Safety, Pharmacokinetics and Efficacy Study of Compound 1 in Subjects Undergoing Bunionectomy

Compound 1 was investigated as a potential therapy for treatment of pain following bunionectomy. Compound 1 was administered (injected and instilled) directly into the postoperative wound during the surgery. This study is a follow-on placebo-controlled efficacy and safety study which includes assessments of wound healing and a PK study in enrolled subjects.

Patients: Adults ages 18 to 75 years, inclusive, who are planning to undergo an elective unilateral bunionectomy (BUNX) and otherwise meet eligibility criteria may be considered for enrollment into the study.

### Criteria:

### Inclusion Criteria:

In the medical judgment of the investigator, be a reasonably healthy adult aged 18-75 years old, inclusive, and American Society of Anesthesiology (ASA) physical Class 1, 2 or 3 at the time of randomization.
Plan to undergo an elective primary unilateral first metatarsal Bunionectomy repair, without collateral procedure or additional surgeries, to be performed under light to moderate sedation with regional anesthesia.
If a male, unless he has a same sex partner, be either sterile (surgically or biologically) or commit to an acceptable method of birth control while participating in the study. The site personnel will provide instructions on what is an acceptable method.
If a female of child-bearing potential (FCBP), must meet all of the following:
   a. Not be pregnant (FCBP must have a negative serum pregnancy test at screening and negative urine pregnancy test before surgery);
   b. No plan to become pregnant or to breast feed during the study; and
   c. Be surgically sterile or at least one year post-menopausal, have a monogamous partner who is surgically sterile, have a same sex partner or (one of the following must apply)
      i. is practicing double-barrier contraception
      ii. is practicing abstinence (must agree to use doublebarrier contraception in the event of sexual activity)
      iii. is using an insertable, injectable, transdermal or combination oral contraceptive approved by the FDA for at least 2 months prior to screening and commits to the use of an acceptable form of birth control while participating in the study.
Have a body mass index ≤40 kg/m².
Be willing and able to sign the informed consent form (ICF) approved by an Institutional Review Board (IRB).
Be willing and able to complete study procedures and pain scales and to communicate meaningfully in English with study personnel and return for outpatient follow up visits as required.
For the portion of subjects who will participate in the PK analyses, be willing to undergo 17 blood draws for PK assessments at various time points during the surgery and the following 24 hours.

### Exclusion Criteria:

In the opinion of the Investigator:
   a. have a concurrent painful condition, other than bunion-related pain, that may require analgesic treatment during the study period or may confound post-surgical pain assessments.
   b. have active skin disease or other clinically significant abnormality at the anticipated site of surgery that could interfere with the planned surgery.
Have a known allergy to chili peppers, capsaicin or the components of Compound 1, bupivacaine HC1, ketorolac, acetaminophen or oxycodone.
As determined by the investigator (with input from the study's medical monitor if requested by the investigator), have a history or clinical manifestation of significant medical, neuropsychiatric or other condition, including an existing arrhythmia, left bundle branch block, myocardial infarction within the prior 6 months, clinically significant abnormal ECG or clinical laboratory test value, that could preclude or impair study participation or interfere with study assessments.
The following are considered disallowed medications:
   a. Be tolerant to opioids defined as those who have been receiving or have received chronic opioid therapy greater than 15 mg of oral morphine equivalents (Table 5) per day for greater than 4 out of 7 days per week over a one-month period within 6 months screening.
   b. Within 1 day prior to surgery and throughout the inpatient period, be taking any capsaicin-containing products, such as dietary supplements or over-the-counter (OTC) preparations, including topical formulations, and prescription medications.
   c. Within the 7 days prior to surgery, be taking any central nervous system (CNS) active analgesic adjunct medication, such as anticonvulsants, antidepressants (such as SNRIs, SSRIs, and tricyclic antidepressants), benzodiazepines, sedative-hypnotics, clonidine and other central alpha-2 agents (e.g., tizanidine), ketamine or muscle relaxants. [Note that SNRIs=Serotonin and norepinephrine reuptake inhibitors and SSRI=Selective serotonin reuptake inhibitors.]
      i. These drugs are permitted if prescribed for non-pain indications and the dose has been stable for at least 30 days prior to surgery. Note that the dose must remain stable throughout the study.
      ii. If the subject is taking centrally- and/or peripherally-acting analgesic medications, such as acetaminophen, NSAIDs, tramadol or opioids, FOR bunion-related pain, the subject may participate in the study if 4(a) above is not applicable and the subject is willing to discontinue these medications 3 days prior to surgery.
      iii. The use of benzodiazepines and the non-benzodiazepines (eszopiclone, ramelteon, zaleplon and zolpidem) are permitted to treat insomnia during the postoperative period.
   d. Within the 7 days prior to the planned surgery and throughout the study, be taking antiarrhythmics except beta-blockers, digoxin, warfarin (see exception below), lithium, or aminoglycosides or other antibiotics for an infection (except for ophthalmic use or for treatment or prophylaxis of postoperative surgical site infections). (Use of warfarin or other agents is allowed, at the investigator's discretion, for DVT prophylaxis after the surgery is completed).
   e. Within the 14 days prior to surgery, be taking parenteral or oral corticosteroids (steroid inhaler for allergy or asthma treatment, topical steroid for a non-clinically significant skin condition not involving the area of surgery or ophthalmic steroids are permissible).
   f. Be on an antianginal, antihypertensive agent or diabetic regimen at a dose that has not been stable for at least 30 days or which is not expected to remain stable while participating in the study.
In the opinion of the Investigator, within the past year have a history of illicit drug use or prescription medicine or alcohol abuse (regularly drinks >4 units of alcohol per day; where a unit=8 oz. beer, 3 oz. wine or 1 oz. spirits).
Have positive results on the alcohol test (breath or saliva) indicative of alcohol abuse or urine drug screen indicative of illicit drug use (unless results can be explained by a current prescription or acceptable over-the-counter medication at screening as determined by the investigator) at screening, and/or prior to surgery.
   a. Note that for those subjects who test positive for tetrahydrocannabinol (THC), if they are willing to abstain from use or consumption of THC-containing products from 3 days prior to surgery to the day 8 visit, they may be allowed to participate in the study. Additionally, it may be permissible for the subject to participate if the results can be explained by a current prescription or acceptable over-the-counter medication as determined by the investigator at screening, and/or prior to surgery.
Have previously participated in a clinical study with Compound 1.
Have participated in another clinical trial or used an investigational product within 30 days or five half-lives (whichever is longer) prior to the planned bunionectomy surgery, or is scheduled to receive an investigational product other than Compound 1 while participating in the study.

### Study Design:

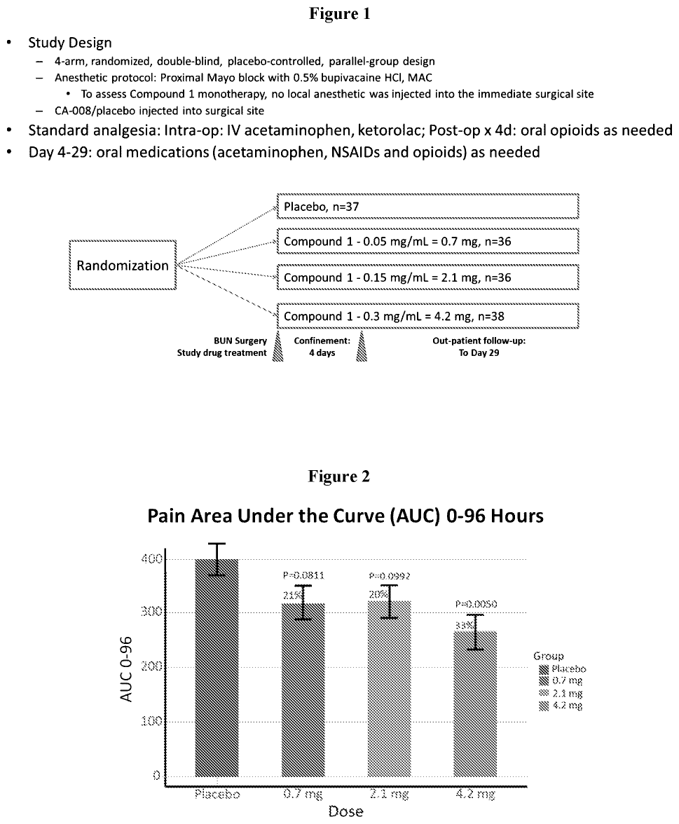

This trial was designed to determine the efficacy of study treatment (Compound 1 vs. placebo) infiltrated/instilled during surgery in treating postoperative pain after a bunionectomy in the setting of multimodal analgesia in addition to a Mayo block with bupivacaine in the first 24 h (FIG. 1).

### Primary Objective:

Evaluate the efficacy of a single intraoperative administration of Compound 1 vs. placebo in subjects undergoing an elective bunionectomy (BUNX).

### Secondary Objectives:

Evaluate the safety and tolerability of a single intraoperative administration of Compound 1 vs. placebo in subjects undergoing an elective BUNX.
Evaluate the PK profile of a single intraoperative administration of Compound 1 vs. placebo in subjects undergoing an elective BUNX.
Identify the minimally effective and optimal doses of Compound 1 to be administered intraoperatively in subjects undergoing an elective BUNX.

Protocol: This Phase 2, multi-center, randomized, double-blind, placebo-controlled, parallel design study evaluated a single dose of one of three Compound 1 dose groups vs. placebo injected during an elective BUNX with a multimodal analgesia regimen including an NSAID and regional Mayo block with bupivacaine. The study was conducted in two parts:
Inpatient period which continued to 96h after completion of study treatment injection (TO) or T96h.
Outpatient period which began on discharge from the inpatient unit through various follow visits to D29±2 (W4) after surgery, or if necessary for ongoing safety assessments, to D36±2 (W5).

Subjects underwent unilateral transpositional first metatarsal osteotomy for the correction of hallux valgus deformity (bunionectomy or BUNX). The surgery was performed under light to moderate sedation anesthesia (what constitutes this level of sedation and the additional anesthetic medications used per discretion of the anesthesiologist). In accordance with standard of care, light to moderate sedation was supplemented with a Mayo block to produce surgical anesthesia and postoperative analgesia. After administering light to moderate sedation, but prior to surgery, performed a Mayo block by infiltrating 0.5% bupivacaine hydrochloride (up to 30 mL total volume, 150 mg) at least 3 cm proximal to the “surgical site”. The Mayo block was performed at 15-30 minutes prior to study treatment dosing. “Surgical site” is defined as the area extending approximately 2-3 cm in all directions (lateral/medial/proximal/distal) from the incision site and surrounding tissues which may be affected by the infiltration of study treatment.

After the surgery, subjects were monitored for 96 hours in an inpatient unit. Safety and efficacy evaluations were performed. After the first week of enrollment, subjects were enrolled for the pharmacokinetic (PK) portion of the study and had samples drawn prior to Compound 1 injection and at various time points over 24h after surgery until the target of 48 PK subjects (12 per treatment group) completed PK assessments. Subjects were required to meet certain pre-specified criteria prior to discharge.

### Administration of Compound 1

Prior to wound closure, study treatment was injected/instilled into the soft tissues and osteotomy surgical sites with a total volume of 14 mL of Compound 1 or placebo as follows:
Compound 1: 3 doses: 0.7 mg, 2.1 mg, and 4.2 mg in 14 mL volume (concentrations: 0.05, 0.15, 0.3 mg/mL)
Placebo: Compound 1 vehicle (identical to active treatment but without Compound 1)
   Study treatment was injected/instilled into the surgical site as follows:
Instilled 2 mL at cut bone sites prior to fixation
Prior to capsule closure, infiltrated the deep soft tissue and area proximal to the capsule with a total of 9 mL (approximately 2.25 mL into each quadrant circumferentially)
Closed the capsule, but using a small gauge catheter infiltrated 2 mL into the closed capsule space
Prior to closure of the subcutaneous tissues and skin, instilled 1 mL to coat all exposed surfaces of the wound.
   T0 is the time that the study treatment injection/instillation is completed.

Following surgery, subjects were transferred to the appropriate recovery unit where they underwent various assessments over the next 96 hours (end of surgery [EOS] to T96h). In addition to the Mayo block noted above, a multimodal analgesia regimen included the following during the immediate postoperative period:
Ketorolac 30 mg IV (a non-steroidal anti-inflammatory drug (NSAID) to be administered intraoperatively before the end of surgery. Per investigator discretion, adjust the dose as necessary for subject age and medical condition.
Acetaminophen 1000 mg IV to be administered intraoperatively before the end of surgery.

After the non-opioid analgesics noted above has been administered, no additional NSAIDs or acetaminophen were administered during the inpatient phase of the study (through T96h). Additionally, the following rescue medication was able to be administered for any moderate to severe breakthrough pain during the inpatient period:
PO oxycodone 5 mg prn q2h in the first 12 hours after surgery for moderate or severe pain (≥4) as reported by subjects using the 0 to 10 numerical rating scale of current pain intensity (NRS).
PO oxycodone 5 mg prn q4h from 12 to 96 hours after surgery for moderate or severe pain (NRS≥4).

Subjects were encouraged to rescue only for moderate pain scores (>4), however rescue was able to be requested at any time and medication will be provided when requested per protocol timing:
Instilled 2 mL at cut bone sites prior to fixation
Prior to capsule closure, infiltrated the deep soft tissue and area proximal to the capsule with a total of 9 mL (approximately 2.25 mL into each quadrant circumferentially)
Closed the capsule, but using a small gauge catheter infiltrated 2 mL into the closed capsule space
Prior to closure of the subcutaneous tissues and skin, instilled 1 mL to coat all exposed surfaces of the wound.

After surgery, subjects were monitored for 96 hours (through T96h) at the trial site as an inpatient. Safety and efficacy evaluations were performed and blood drawn for PK assessments (for the PK study). Subjects were required to meet standard pre-specified criteria for discharge from the unit. Topical ice packs or cooling treatments were prohibited during the inpatient period for use on the foot near the surgical site. Such treatments were allowed to treat conditions remote from the surgical site. After discharge, topical ice packs or cooling treatments were able to be used over the surgical site or elsewhere.

After completing the assessments through T96h hours after study treatment administration and prior to discharge from the inpatient unit, reviewed with the subject the use of a diary for at-home use to record pain assessments and medication use (including pain medication) at home. Additionally, obtained the subject's global assessment of satisfaction with study treatment (patient global evaluation or PGE) and the investigator's global evaluation (IGE) prior to discharge. Finally, instructed subjects to return to the study center on D8±1 for a follow-up assessment.

### Study Results:

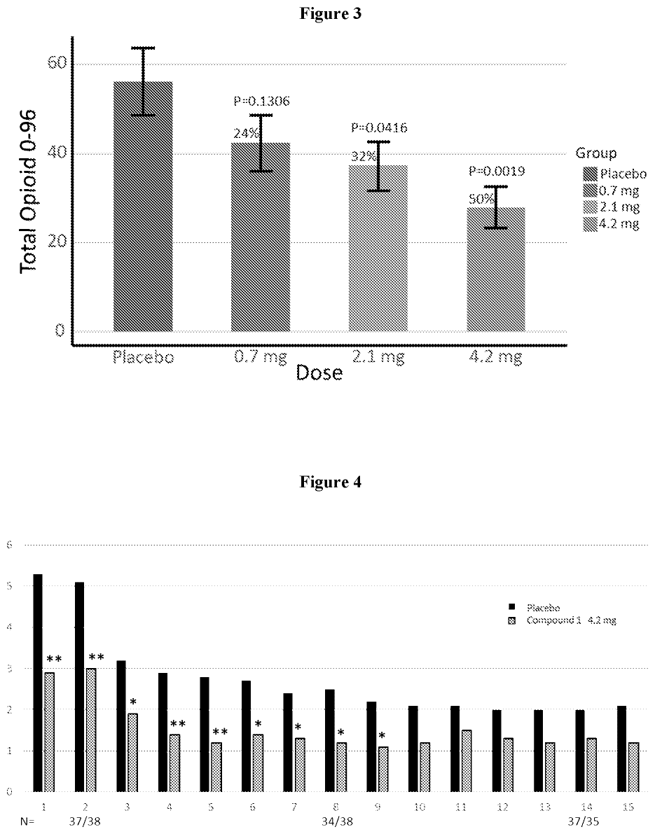

Compound 1 demonstrated durable analgesic benefit at specific concentrations and doses in randomized controlled trials. A single administration of Compound 1 during surgery to patients undergoing bunionectomy resulted in long lasting reductions of pain and opioid consumption. The maximum analgesic effect was seen at 4.2 mg (concentration of 0.3 mg/mL). A dose response was demonstrated for concentration of Compound 1. At a concentration of 0.15 mg/mL we begin to see a trend in pain reduction and statistically significant reductions in opioid consumption (FIG. 2). At a dose of 4.2 mg (0.3 mg/mL) Compound 1 achieved a statistically significant reduction in pain. In addition, at a doses of 2.1 mg (0.15 mg/mL) and 4.2 mg (0.3 mg/mL) Compound 1 achieved a statistically significant reduction in opioid consumption (FIG. 3). An additional finding is the rate of opioid free subjects. Moreover, the analgesic effect of Compound 1 persisted to at least Day 15 (FIG. 4).

### Example 2: A Two-Part, Phase 1/2, Randomized, Double-blind, Placebo-Controlled, Adaptive Safety, Pharmacokinetics, and Preliminary Efficacy Study of Compound 1 in Patients Undergoing Total Knee Arthroplasty (TKA)

Compound 1 was investigated as a potential therapy for treatment of pain following TKA surgery.

Patients: Adults 18 to 80 years of age, inclusive, who are planning to undergo an elective unilateral total knee arthroplasty (replacement) (TKA) and who otherwise meet eligibility criteria may be considered for enrollment into the study.

### Criteria:

### Inclusion Criteria:

Plan to undergo an elective primary unilateral TKA under spinal anesthesia with sedation, without collateral procedure or additional surgeries.
Approriate candidate for spinal anesthesia, including no contraindications, no anatomical constraints.
Adults 18-80 years of age, inclusive.
American Society of Anesthesiology (ASA) physical Class 1, 2, or 3 at the time of randomization.
If a male, unless he has a same sex partner, be either sterile (surgically or biologically) or commit to an acceptable method of birth control while participating in the study. The site personnel will provide instructions on what is an acceptable method.
If a female of child-bearing potential (FCBP), must meet all of the following:
   a. Not be pregnant (FCBP must have a negative serum pregnancy test at screening and negative urine pregnancy test before surgery);
   b. No plan to become pregnant or to breast feed during the study; and
   c. Be surgically sterile or at least one year post-menopausal, have a monogamous partner who is surgically sterile, have a same sex partner or (one of the following must apply)
      i. is practicing double-barrier contraception
      ii. is practicing abstinence (must agree to use doublebarrier contraception in the event of sexual activity)
      iii. is using an insertable, injectable, transdermal or combination oral contraceptive approved by the FDA for at least 2 months prior to screening and commits to the use of an acceptable form of birth control while participating in the study.
Have a body mass index ≤42 kg/m².
Be willing and able to sign the informed consent form (ICF) approved by an Institutional Review Board (IRB).
Be willing and able to complete study procedures and pain scales and to return for outpatient follow up visits as required.
Be willing and able to avoid foods containing capsaicin for 24 hours prior to surgery.

### Exclusion Criteria:

In the opinion of the Investigator:
   a. have a concurrent painful condition, other than bunion-related pain, that may require analgesic treatment during the study period or may confound post-surgical pain assessments.
   b. moderate to severe chronic neuropathic pain or signs of central sensitization.
   c. active skin disease or other clinically significant abnormality at the anticipated site of surgery that could interfere with the planned surgery.
The patient is tolerant to opioids (opioid-tolerant) defined as someone who has been receiving or has received chronic opioid therapy (use within the 2 weeks prior to surgery and duration of use >4 weeks) greater than 15 mg of oral morphine equivalents per day for greater than 3 out of 7 days per week over a one-month period within 6 months of screening.
The patient has a known allergy (or contraindication) to any of the following: chili peppers, capsaicin or the components of Compound 1, ropivacaine hydrochloride (HC1), ketorolac, acetaminophen, fentanyl, hydromorphone, morphine, oxycodone, or celecoxib.
As determined by the Investigator (with input from the study's medical monitor if requested by the Investigator), the patient has a history or clinical manifestation of significant medical, neuropsychiatric, or other condition, including a clinically significant existing arrhythmia, left bundle branch block or abnormal electrocardiogram (ECG), myocardial infarction, or coronary arterial bypass graft surgery within the prior 12 months, significant abnormal clinical laboratory test value, or known bleeding abnormality that could preclude or impair study participation or interfere with study assessments.
Use of the following disallowed medications:
   a. Within 1 day prior to surgery and throughout the inpatient period, taking or using any capsaicin-containing products, such as dietary supplements or over-the-counter (OTC) preparations, including topical formulations, and prescription medications.
   b. Within the 7 days prior to surgery, taking any central nervous system (CNS) active agent as an analgesic adjunct medication, such as anticonvulsants, antidepressants (such as SNRIs, SSRIs, and tricyclic antidepressants), benzodiazepines, sedative-hypnotics, clonidine and other central alpha-2 agents (e.g., tizanidine), ketamine, or muscle relaxants.
      i. These drugs are permitted if prescribed for non-pain indications and the dose has been stable for at least 30 days prior to surgery. Note that the dose must remain stable throughout the study.
      ii. If the patient is taking centrally- and/or peripherally-acting analgesic medications, such as acetaminophen, NSAIDs, tramadol, or opioids, for knee-related pain, the patient may participate in the study if not opioid-tolerant (defined above) and the patient is willing to discontinue these medications 3 days prior to surgery. Note that (a) baby aspirin (81 mg/day) for cardiovascular prophylaxis or (b) regular or enteric-coated aspirin (up to 325 mg given up to twice daily) for venous thrombo-embolism prophylaxis is allowed during the study.
   c. Within the 7 days prior to the planned surgery and throughout the study, taking (a) antiarrhythmics (except beta-blockers and digoxin); (b) warfarin or other anticoagulants (see exception below); (c) lithium; or (d) aminoglycosides or other antibiotics for an infection (except for ophthalmic use).
   d. Within the 14 days prior to surgery, be taking parenteral or oral corticosteroids (steroid inhaler for allergy or asthma treatment, topical steroid for a non-clinically significant skin condition not involving the area of surgery or ophthalmic steroids are permissible).
   e. Be on an antianginal, antihypertensive agent or diabetic regimen at a dose that has not been stable for at least 30 days or which is not expected to remain stable while participating in the study.
In the opinion of the Investigator, within the past year have a history of illicit drug use or prescription medicine or alcohol abuse (regularly drinks >4 units of alcohol per day; where a unit=8 oz. beer, 3 oz. wine or 1 oz. spirits).
The patient has a disqualifying positive urine drug screen or alcohol breath/saliva test during screening or check-in.
Have previously participated in a clinical study with Compound 1.
Have participated in another clinical trial or used an investigational product within 30 days or five half-lives (whichever is longer) prior to the planned bunionectomy surgery, or is scheduled to receive an investigational product other than Compound 1 while participating in the study.

### Study Design:

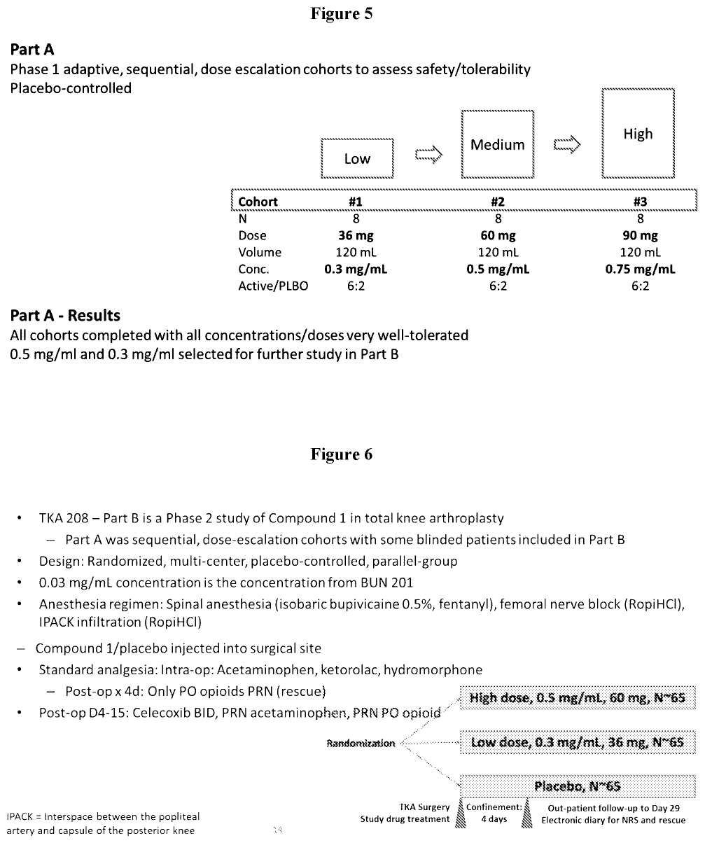

This is a two-part, Phase 1/2, randomized, double-blind, placebo-controlled study. In Part A, 2 or more exploratory sequential dose-escalation cohorts will be evaluated, each with administration of a single dose of Compound 1 or placebo infiltrated/instilled during surgery in patients undergoing TKA (FIG. 5). In Part B, 2 or 3 active dose levels of Compound 1 will be evaluated compared to placebo in a randomized, double-blind, parallel-group design (FIG. 6).

### Primary Objective (Part A):

Evaluate the safety and tolerability of a single intraoperative administration of either Compound 1 or placebo in patients undergoing an elective TKA.

### Secondary Objectives (Part A):

Evaluate the PK profile of a single intraoperative administration of Compound 1 in patients undergoing an elective TKA.
Evaluate, preliminarily, the activity of Compound 1 on reported pain, opioid consumption, and patient-reported outcomes (PROs).
Determine the dose(s) for testing in Part B.

### Primary Objective (Part B):

Evaluate, during a specified post-operative time interval, the efficacy of 2 or 3 selected doses of Compound 1 on reported pain in patients undergoing an elective TKA.

### Secondary Objectives (Part B):

Evaluate the efficacy of 2 or 3 selected doses of Compound 1 on reported pain in patients undergoing an elective TKA during additional specified post-operative time intervals.
Evaluate the effect of Compound 1 on opioid consumption.
Evaluate the effect of Compound 1 on PROs.
Evaluate, preliminarily, the effect of Compound 1 on performance-based outcome measures (PBOMs), including knee range of motion (ROM) .
Evaluate the safety and tolerability of Compound 1 or placebo in patients undergoing an elective TKA.
Evaluate the PK profile of a single intraoperative administration of Compound 1 in patients undergoing an elective TKA.

### Dosage Groups:

### Part A

1. Cohort #1: total N=8 patients randomized 3:1 Compound 1 36 mg in 120 mL (0.3 mg/mL) or placebo 120 mL
2. Cohort #2: total N=8 patients randomized 3:1 Compound 1 60 mg in 120 mL (0.5 mg/mL) or placebo 120 mL
3. Cohort #2: total N=8 patients randomized 3:1 Compound 1 90 mg in 120 mL (0.75 mg/mL) or placebo 120 mL

### Part B

1. Cohort #1: total N=˜97 patients randomized 2:1 Compound 1 36 mg in 120 mL (0.3 mg/mL) or placebo 120 mL
2. Cohort #2: total N=˜97 patients randomized 2:1 Compound 1 60 mg in 120 mL (0.5 mg/mL) or placebo 120 mL

Subjects underwent unilateral total knee arthroplasty (replacement) (TKA). The surgery was performed under spinal anesthesia (isobaric bupivacaine [preservative-free] 0.5% 12-15 mg, and fentanyl 25 mcg) with adequate supplemental sedation, typically with midazolam (up to 5 mg), fentanyl (up to 100 mcg), and propofol infusion, but anesthesia and sedation per institutional guidelines. If the patient appeared to have an inadequate level of anesthesia from the spinal anesthetic (“failed spinal anesthetic”), the patient received a general anesthetic with either an inhaled anesthetic or propofol infusion with or without nitrous oxide. In addition, patients received ropivacaine hydrochloride (RopiHCl) delivered as follows:
Prior to start of surgery under ultrasound guidance:
   Femoral nerve block: RopiHCl 0.5%, 12 mL (60 mg)
IPACK infiltration: RopiHCl 0.2%, 20 mL (40 mg)

### And

Immediately after surgical incision:
   Periarticular infiltration: RopiHCl 0.2%, 50 mL (100 mg)
   Periarticular infiltration was performed using a 22-gauge needle as follows:
   After the surgical incision and prior to the arthrotomy, infiltrated 25 mL of RopiHCl into the tissue adjacent to and along the planned arthrotomy incision site.
   After the arthrotomy and after the initial dissection but prior to bony resection/cuts, infiltrated 25 mL of RopiHCl into the residual soft tissue envelope of the distal femur and proximal tibia that had been surgically debrided (eg, the fat pad, subperiosteal dissection, synovium, etc).

In general, the intent was to deliver local anesthetic into the surgical site “on the way in” (upon adequate exposure of and prior to incision of target tissues). Conversely, study drug (active or placebo) was administered to target tissues “on the way out” (at the time of device implantation and surgical closure).

### Administration of Compound 1

The initial total study drug volume administered per patient was 120 mL. This initial 120 mL of study treatment administered per patient in the first study cohort was delivered during the surgical procedure in 4 divided aliquots. The initial allocation was:
(a) a total of 15 mL instilled onto the cut surfaces following each osteotomy;
(b) 30 mL infiltrated prior to hardware placement;
(c) 60 mL infiltrated following hardware placement; and
(d) 15 mL instilled following closure of the joint capsule.

Following surgery, patients were transferred to the PACU where they underwent an assessment of safety and efficacy over the next 96 (±4) hours. T0 is the time of admission into the PACU (as recorded in notes by the PACU nurse). The schedule of assessments was as follows:
Patients with inadequately controlled pain may request rescue at any time during T0 to D5; however, patients will be encouraged to receive rescue medication only with an NRS ≥4.
Pain intensity (assessed with NRS).
During the inpatient stay, NRS at rest beginning with the PACU admission was able to be assessed once the patient is awake. T0 is the time of admission into the PACU (as recorded in notes by the PACU nurse). If the patient was able to provide responses, obtained NRS scores at T0 plus 1 hour (T1), T0 plus 2 hours (T2, etc.), T4, T6, T8, T12, T16, T20, T24, and every 4 hours thereafter (if awake at time of assessment) until discharge from the inpatient unit. Time windows: ±5 minutes for T1 and T2; ±15 minutes for T4 onward. Scheduled NRS scores must be recorded regardless of timing of pre-rescue medication NRS scores and administration of rescue medication. The actual time of all NRS scores must be recorded, i.e., not the nominal time.
During the inpatient stay, evoked NRS twice daily after physical therapy (most preferred) or ambulation for approximately 10 yards (preferred) or transfers to/from bed or wheelchair. Obtained these NRS scores after morning physical therapy or ambulation at 10:00 AM (±1 h) and after afternoon physical therapy or ambulation at 4:00 PM (±1 h).
During the inpatient stay, pain scores were able to be skipped between the hours of midnight and 6:00 AM, but the patient could not miss two consecutive assessments. The T12, T24, T48, T72, and T96 assessments were completed even if the patient must be awakened at these times.
During the inpatient stay, an additional NRS assessment was obtained within 5 minutes prior to IV rescue medication administration and within 15 minutes prior to oral rescue medication administration.

After completing the assessments through T96, the diary for at-home use will be reviewed and patients will be discharged from the study center with the diary to record pain assessments and pain medication at home. Patients will be provided routine standard of care for pain management after discharge from the study center.

Patients were given a prescription for 20 tablets of acetaminophen 325 mg/oxycodone 5 mg (Percocet) one or two tablets PO Q 4-6 h PRN moderate-severe pain (i.e., NRS 5-10). Document the number of tablets prescribed and also document any additional prescriptions required if the patient's pain continues to be an issue requiring additional opioid medication.

### Study Results:

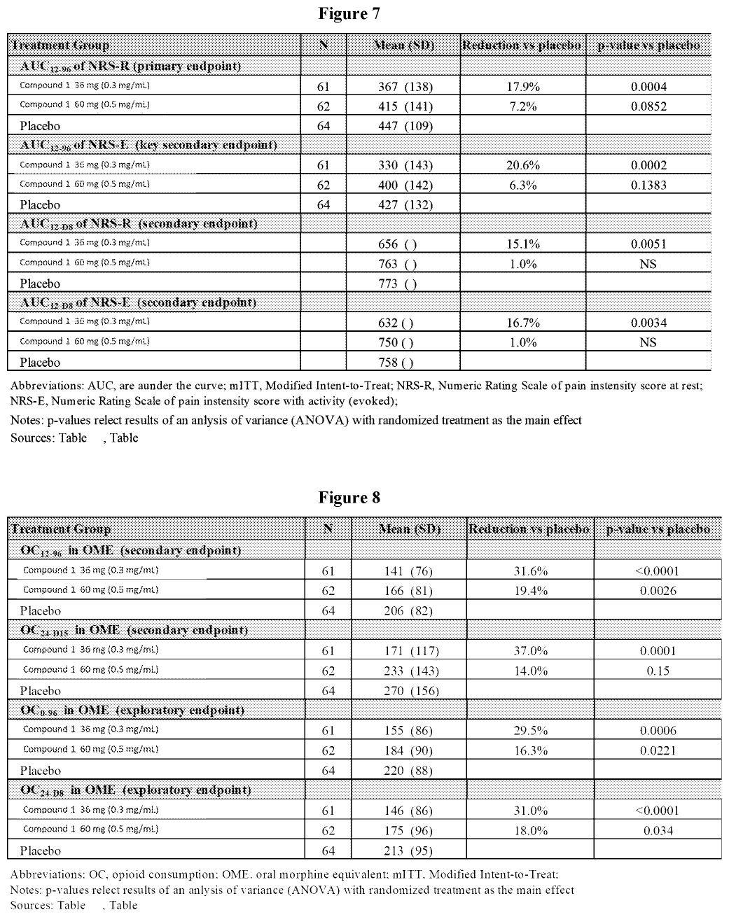

Compound 1 at a dose of 36 mg (0.3 mg/mL) demonstrated long lasting reductions in pain in patients undergoing knee replacement (TKA) (FIG. 7). A higher dose of Compound 1, 60 mg (0.5 mg/mL), demonstrated lower analgesic activity which was of a shorter duration. In addition, at a dose of 36 mg (0.3 mg/mL) Compound 1 achieved a significant reduction in opioid consumption (FIG. 8).

### Example 3: A Phase 2, Randomized, Double-blind, Placebo-Controlled Efficacy, Pharmacokinetics and Safety Study of Compound 1 in Subjects Undergoing Ventral Hernia Repair

Compound 1 was investigated as a potential therapy for treatment of pain in subjects undergoing surgery to repair a ventral hernia.

Patients: Adults 18 to 80 years of age, inclusive, who are planning to undergo an elective open laparotomy for ventral hernia repair (VHR), with retromuscular, preperitoneal mesh repair (i.e., Rives-Stoppa technique or equivalent), with or without laparoscopic assistance, and who otherwise meet eligibility criteria.

### Criteria:

### Inclusion Criteria:

1. Plan to undergo an elective, primary, open laparotomy with VHR, with retromuscular, preperitoneal synthetic (polypropylene) mesh placement, with midline fascial reconstruction (i.e., Rives-Stoppa technique or equivalent) and with optional laparoscopic assistance under general anesthesia with sedation, without collateral procedure or additional surgeries. Ventral hernia should not be longer than 8 cm in length.
2. Appropriate candidate for TAP block, including no contraindications, no anatomical constraints. Note: TAP block may replace or supplement rectus sheath block if rectus sheath block inadequate.
3. Adults 18-80 years of age, inclusive.
4. American Society of Anesthesiology (ASA) physical Class 1, 2, or 3 at the time of randomization (Section 17.2, Appendix B).
5. If a male, be either sterile (surgically or biologically) or commit to an acceptable method of birth control while participating in the study. The site personnel will provide instructions on what is an acceptable method.
6. If a female, must meet all of the following:
   a. Not be pregnant (FCBP must have a negative serum pregnancy test at screening and negative urine pregnancy test before surgery);
   b. No plan to become pregnant or to breast feed during the study; and
   c. Be surgically sterile or at least one year post-menopausal, have a monogamous partner who is surgically sterile, have a same sex partner or (one of the following must apply)
      i. is practicing double-barrier contraception
      ii. is practicing abstinence (must agree to use doublebarrier contraception in the event of sexual activity)
      iii. is using an insertable, injectable, transdermal or combination oral contraceptive approved by the FDA for at least 2 months prior to screening and commits to the use of an acceptable form of birth control while participating in the study.
7. Have a body mass index ≤40 kg/m².
8. Be willing and able to sign the informed consent form (ICF) approved by an Institutional Review Board (IRB).
9. Be willing and able to complete study procedures and pain scales and to return for outpatient follow up visits as required.
10. Be willing and able to avoid foods containing capsaicin for 24 hours prior to surgery.

### Exclusion Criteria:

1. In the opinion of the Investigator, the patient has:
   a. a concurrent chronic painful condition (i.e., daily pain) that may require analgesic treatment during the study period or may confound post-surgical pain assessments;
   b. active skin disease or other clinically significant abnormality at the anticipated site of surgery that could interfere with the planned surgery.
2. The patient has taken opioids more than twice per week in any week within 6 months of screening or has used any opioids during the 2 weeks prior to surgery.
3. The patient has a known allergy (or contraindication) to any of the following: chili peppers, capsaicin or the components of Compound 1, propofol, bupivacaine hydrochloride (HCl), midazolam, inhaled anesthetic, nitrous oxide, ondansetron, acetaminophen, fentanyl, hydromorphone, morphine, oxycodone, or celecoxib.
4. As determined by the Investigator (with input from the study's medical monitor if requested by the Investigator), the patient has a history or clinical manifestation of significant medical, neuropsychiatric, or other condition, including a clinically significant existing arrhythmia, left bundle branch block or abnormal ECG, myocardial infarction, or coronary arterial bypass graft surgery within the prior 12 months, significant abnormal clinical laboratory test value, or known bleeding abnormality that could preclude or impair study participation or interfere with study assessments.
5. Use of the following disallowed medications:
   a. Within 1 day prior to surgery and throughout the inpatient period, taking or using any capsaicin-containing products, such as dietary supplements or over the counter (OTC) preparations, including topical formulations, and prescription medications.
   b. Within the 7 days prior to surgery, taking any central nervous system active agent as an analgesic adjunct medication, such as anticonvulsants (e.g., gabapentin), antidepressants (such as SNRIs, SSRIs, and tricyclic antidepressants), benzodiazepines, sedativehypnotics, clonidine and other central alpha-2 agents (e.g., tizanidine), ketamine, or muscle relaxants.
   i. These drugs are permitted if prescribed for non-pain indications and the dose has been stable for at least 30 days prior to surgery. Note that the dose must remain stable throughout the study.
   ii. If the patient is taking centrally- and/or peripherally acting analgesic medications, such as acetaminophen, nonsteroidal antiinflammatory drug (NSAIDs), or pregabalin, the patient may participate in the study if the patient is willing to discontinue these medications 3 days prior to surgery. Note that (a) baby aspirin (81 mg/day) for cardiovascular prophylaxis or (b) regular or enteric-coated aspirin (up to 325 mg given up to twice daily) for venous thrombo-embolism prophylaxis is allowed during the study.
   c. Within the 7 days prior to the planned surgery taking (a) antiarrhythmics (except beta-blockers and digoxin); (b) warfarin or other anticoagulants (see exception above); (c) lithium; (d) aminoglycosides or other antibiotics for an infection (except for ophthalmic use); or (e) medical (or other) regular marijuana use.
   d. Within the 14 days prior to surgery, taking parenteral or oral corticosteroids (steroid inhaler for allergy or asthma treatment, topical steroid for a nonclinically significant skin condition not involving the area of surgery, or ophthalmic steroids are permissible).
   e. Taking an antianginal, antihypertensive agent or diabetic regimen at a dose that has not been stable for at least 30 days or which is not expected to remain stable while participating in the study.
6. In the opinion of the Investigator, within the past year, the patient has a history of illicit drug use or prescription medicine or alcohol abuse (regularly drinks >4 units of alcohol per day; where a unit=8 oz. beer, 3 oz. wine, or 1 oz. spirits).
7. The patient has a disqualifying positive urine drug screen or alcohol breath/saliva test during screening or check-in (See Section 10.3.14).
8. The patient has previously participated in a clinical study with Compound 1.
9. The patient has participated in another clinical trial or used an investigational product within 30 days or 5 half-lives (whichever is longer) prior to the planned surgery or is scheduled to receive an investigational product other than Compound 1 while participating in the study.

### Study Design:

This is a three-part, Phase 1/2, randomized, double-blind, placebo-controlled, adaptive, pharmacokinetics (PK), and safety study of Compound 1 in patients undergoing VHR.
In Part A, Compound 1 15 mg will be administered in an open-label exploration of different delivery techniques. The objective is to determine the safety, feasibility, PK and appropriateness of administration of a single dose of Compound 1 infiltrated/instilled during surgery in patients undergoing VHR. It is expected that 8-12 patients will be enrolled in Part A, but it may be up to 16 patients so that at least 8 patients receive the optimal delivery technique which will allow a formal safety assessment of the 15 mg dose of study drug.
In Part B, if the formal safety assessment from Part A of Compound 1 15 mg is favorable, then the active study drug dose will be increased to 30 mg delivered as 100 mL of a 0.3 mg/mL solution. Using the general delivery technique identified in Part A, this dose, concentration and volume will be assessed compared to a placebo in a pilot, double-blind, randomized, parallel-group design (see Figure). The objective of Part B is to determine preliminarily the tolerability of planned study drug administration as well as the pain profile of the placebo control group. It is expected that approximately 24 patients (up to 32) will be enrolled in Part B. The results of Part B will be unblinded for analysis prior to the initiation of Part C.
In Part C, the active dose level of Compound 1 from Part B will be evaluated compared to placebo in a larger randomized, double-blind, parallel-group design to evaluate efficacy and safety. It is expected that ˜100 patients will be enrolled in Part C to bring the total number of patients enrolled in the study to up to ˜150.
For each patient, the study will be conducted in two periods:
Inpatient period which continues from check-in on D1 until discharge (4 days or 96 hours [h] following surgery [T96±4 h], [D5]). Discharge may be delayed, if needed, for medical reasons.
Outpatient period which begins on discharge from the inpatient unit through follow up visits to D29 +2 days. Note that additional follow up visits may occur at any time or even after D29 to follow AEs to resolution or to establish a new baseline.
During the inpatient period, patients will undergo VHR including study drug treatment (Compound 1 or placebo) followed by serial assessments of safety, PK, and drug effect, in particular focusing on reported pain and need for analgesia. During the outpatient period, patients will have serial assessments of safety and drug effect.

### Primary Objective (Part A):

Evaluate the safety, tolerability and feasibility of a single intraoperative administration of Compound 1 15 mg in patients undergoing an elective VHR.

### Secondary Objectives (Part A):

Evaluate the PK profile of a single intraoperative administration of Compound 1 15 mg in patients undergoing an elective VHR.

### Primary Objective (Part B):

Evaluate the safety, tolerability and feasibility of a single intraoperative administration of Compound 1 30 mg in patients undergoing an elective VHR.

### Secondary Objectives (Part B):

Determine the pain profile of patients undergoing an elective VHR.
Determine the appropriateness of progression to Part C.
Assessment of opioid consumption.
Evaluate the PK profile of a single intraoperative administration of Compound 1 30 mg in patients undergoing an elective VHR.
Explore the relationship between Compound 1 and its metabolite plasma concentrations and electrocardiogram (ECG) QT interval using a concentration-QT (cQT) analysis.

### Primary Objective (Part C):

Evaluate the efficacy of Compound 1 on reported pain in patients undergoing an elective VHR during a specified post-operative time interval.

### Secondary Objectives (Part C):

Evaluate the efficacy of Compound 1 on reported pain in patients undergoing an elective VHR during additional specified post-operative time intervals.
Evaluate the effect of Compound 1 on opioid consumption.
Evaluate the effect of Compound 1 on patient-reported outcomes (PROs)
Evaluate, preliminarily, the effect of Compound 1 on performance-based outcome measures (PBOMs).
Evaluate the safety and tolerability of Compound 1 or placebo in patients undergoing an elective VHR.

### Study Treatment and Dosing Schedule:

Study treatment was administered intraoperatively as a single administration via infiltration/instillation into the “surgical site” prior to wound closure. In general, the intent was to deliver local anesthetic into the surgical site “on the way in” (upon adequate exposure of and prior to incision/dissection of target tissues). Conversely, study drug (active or placebo) delivery to the same and other areas was administered by infiltration (injected) and/or instillation/irrigation (dripped) to the surgical site “on the way out” (prior to and at the time of surgical closure). The “surgical site” is defined as the area extending approximately at least 2-3 cm in all directions (lateral/medial/proximal/distal/deep) from the margins of substantially all tissue traumatized by the surgical procedure, i.e., all tissue dissected, cut, electrocauterized (bovied), sutured or tacked, including both deep and superficial areas. The surgical site include sthe full area of surgical mesh placement.

### Number of Participants

Part A—Open-label, feasibility and safety assessment:
   N=At least 8 with an acceptable range of up to 16
Part B—Double-blind, placebo-controlled pilot: o Expected N=24 with an acceptable range of up to N=32 (randomized 1:1 active to placebo)
Part C—Double-blind, placebo-controlled efficacy:
   In Part C, a total of up to N=˜100 patients will be randomized in a 1:1 ratio to active or placebo to bring the total number of patients enrolled in the study to up to ˜150.

### Dose Groups:

In Part A, Compound 1 15 mg (50 ml of a 0.3 mg/mL solution) was delivered and used to explore the optimal technique of delivery, i.e., location and volumes of delivery to the different areas and layers of the surgical procedure. Parts B and C, Compound 1 30 mg (100 mL of a 0.3 mg/mL concentration) or blinded placebo was delivered using the general technique identified in Part A.

### Injection of Study Treatment:

Part A: The initial total study drug volume administered per patient was 50 mL delivered during the surgical procedure in divided aliquots.
Deep midline layer—10 mL
Mesh layer—30 mL
Anterior layer—10 mL
   Parts B and C: The expected allocation will be as follows, equating to a total study drug volume of 100 mL (details follow in the protocol):
Deep midline layer—25 mL
Mesh layer—50 mL
Anterior layer—25 mL (up to 5 mL of this volume may be delivered to the laparoscopy port(s))

### Preoperative, Anesthesia and Perioperative Care:

At check-in (pre-operatively), at least 1-2 hours before surgery:
Celecoxib 200 mg orally (PO)
Acetaminophen 1000 mg PO
   After these medications were administered, no additional non-opioid analgesics were administered during the inpatient phase (through T96).
   The surgery was performed under general anesthesia with the following guidelines:
Inhaled anesthetic or propofol infusion with or without nitrous oxide (N20).
Adequate optional premedication typically with midazolam (up to 5 mg), fentanyl (up to 100 mcg), more can be given if indicated
Supplemental anesthesia and sedation will be per institutional guidelines Intra-operative titration of intravenous (IV) fentanyl, including during emergence, will be per institutional guidelines.
   In addition, patients received bupivacaine hydrochloride (BupiHCl) 175 mg delivered as follows:
Prior to start of surgery under ultrasound guidance:
   BupiHCl 0.25%, 30 mL (75 mg) diluted with 10 mL of normal saline to a volume of 40 mL delivered as a rectus block with 20 mL on each side.
Immediately before/after surgical incision:
   BupiHCl 0.25%, 40 mL (100 mg), may be diluted in up to 110 mL of normal saline to a total volume of not more than 150 mL: Infiltration of abdominal wall, anterior rectus abdominis sheath, posterior rectus abdominis sheath, other fascia, peritoneum (including circumferential areas of intended mesh placement)
      Standard supplemental anesthetic and perioperative care was provided per institutional guidelines. This typically included (but not be limited to) steps to ensure preservation of intraoperative normothermia (including the use of preoperative and intraoperative forced-air warming) and deep venous thrombosis prophylaxis.

### Intraoperative Analgesia and Care:

Patients received general anesthesia and bilateral rectus blocks. During surgery, patients received the following:
3-500 mL IV fluid, more can be given if indicated
Ondansetron 4 mg IV
   Within 15 minutes prior to the end of surgery, administer the following:
IV hydromorphone, 0.5 mg
Note: IV morphine 2.0 mg was used if IV hydromorphone is not available
   Patients who develop clinically significant hemodynamic instability or other anesthesia complication prior to study drug administration should not receive study drug; in Parts A and B, these patients will be replaced. Replacement patients will be assigned the same treatment as the original patient. A ‘replacement’ randomization list matching that of the main list will be created to facilitate this process.
   Rescue Medication during the Inpatient Period:
   Patients were encouraged to use rescue medication only for moderate-to-severe pain (NRS≥4); however, rescue medication may be requested at any time (i.e., even when NRS<4) and medication will be provided when requested. Conversely, patients may refuse rescue medication even when the NRS is ≥4. Time intervals for all rescue medication use are guidelines that can be modified by Investigator discretion.
From the time of post-anesthesia care unit (PACU) discharge through T12:
   Administer IV hydromorphone 0.5 mg, Q15 minutes PRN for pain of NRS≥4.
   Note: IV morphine 2.0 mg can be used if IV hydromorphone is not available
After T12-48:
   Administer PO oxycodone 10 mg, Q 4 hours (h) PRN for pain NRS≅4.
   Only PO oxycodone may be used as rescue. If a patient still requires IV opioid rescue after T12, then the patient will revert to IV opioid analgesia management per institutional guidelines. These patients will still also be followed for NRS, safety, and all other assessments.
After T48-96:
   Administer PO oxycodone 5 mg, Q 4 h PRN pain NRS 5-10.
   Only PO oxycodone may be used as rescue. If a patient still requires IV opioid rescue after T12, then the patient will revert to IV opioid analgesia management per institutional guidelines. These patients will still also be followed for NRS, safety, and all other assessments.

### Postsurgical Care:

After surgery, patients were transferred to the post-anesthesia care unit (PACU) where patients were monitored for at least 90 minutes during which time pain assessments began once the patient is awake. T0 is the time of admission into the PACU (as recorded in notes by the PACU nurse). The time of extubation was recorded, if applicable. Patients used the 0 to 10 numerical rating scale (NRS) to report their current pain intensity multiple times per day during the remainder of the inpatient part of the study. Rescue medication was only be administered upon request, i.e., independent of the currently reported pain score. That is, an NRS pain score alone does not trigger rescue medication administration. If the patient reports pain spontaneously and requests analgesia at an unscheduled time (i.e., PRN), an NRS should be used to record the pain present at that time. This unscheduled NRS must be recorded just prior to administration of any PRN analgesia, i.e., within 5 minutes prior to any PRN IV analgesic treatment and within 15 minutes prior to any PRN oral analgesic treatment. NRS scores for PRN pain medication will not replace the recording of scheduled NRS scores.
Medication for moderate to severe pain (NRS≥4) was administered upon request as follows:
From T0 to T25 minutes:
   IV fentanyl 50 mcg, every (Q) 5 minutes for pain NRS≥4
From T26 minutes to PACU discharge:
   IV hydromorphone 0.5 mg, Q 10 minutes for pain NRS≥4
   Note: IV morphine 2.0 mg can be used if IV hydromorphone is not available
      After discharge from the PACU, patients were followed through T96 (±4 h) as inpatients in the inpatient unit where safety and activity/efficacy evaluations were performed. Patients were required to meet standard criteria for discharge to outpatient status. Patients were monitored as outpatients after discharge from the inpatient unit through D29+2 days for various safety and efficacy assessments, and later if necessary, for safety follow up.

### Activity/Efficacy Parameter Assessments:

1. NRS scores were assessed as follows:
During the inpatient stay, NRS at rest beginning with the PACU admission was assessed once the patient is awake. T0 is the time of admission into the PACU (as recorded in notes by the PACU nurse). Obtain NRS scores T0 plus 1 hour (T1), T0 plus 2 hours (T2), T4, T6, T8, T12, T16, T20, T24, and every 4 hours thereafter (if awake at time of assessment) until discharge from the inpatient unit. Time windows: ±5 minutes for T1 and T2; ±15 minutes for T4 onward. Scheduled NRS scores were recorded regardless of timing of pre-rescue medication NRS scores and administration of rescue medication.
During the inpatient stay, as soon as feasible, obtained evoked NRS twice daily after 3 maneuvers: (a) coughing 3 times, and (b) sitting up from the supine position into a standardized position (both legs dangling on the side of the bed), and (c) ambulation for approximately 10 yards (30 feet). Obtained these NRS scores in the morning at 10:00 AM (±1 h) and in the afternoon at 4:00 PM (±1 h).
During the inpatient stay, pain scores could be skipped between the hours of midnight and 6:00 AM, but the patient did not miss two consecutive assessments. The T12, T24, T48, T72 and T96 assessments were completed even if the patient must be awakened at these times.
During the inpatient stay, an additional NRS assessment was obtained within 5 minutes prior to IV rescue medication administration and within 15 minutes prior to oral rescue medication administration.
During the outpatient period (after T96 and through D15), an additional NRS assessment was obtained prior to oral rescue medication administration. If entered in the electronic diary, the timing of the additional NRS assessment was assumed to be coincident with the use of rescue medication. If not entered in the electronic diary, the additional NRS assessment should be within 15 minutes prior to use of rescue medication to be used for imputation.
During the outpatient period (after T96 and through D15), the patient was instructed to document their NRS scores twice daily at 11:00 AM (±1 h) and 7:00 PM (±1 h) (a) at rest, (b) after coughing 3 times, (c) after sitting up from the supine position into a standardized position (both legs dangling on the side of the bed), and (d) during ambulation for approximately 10 yards. Patient instructed to:
   Obtain the morning NRS assessments;
   Obtain the evening NRS assessments;
   Opioid and non-opioid rescue medication (dose, date, time) were recorded in the diary through D15, including a pre-rescue NRS score.
      2. Daily opioid consumption (specific drug, dose, date, time) was recorded during the inpatient and outpatient periods (through D15) to allow calculation of total opioid consumption (OC) and daily opioid (rescue medication) consumption in OMEs (oral consumption in oral morphine equivalents).
Document daily use of opioid analgesia during the outpatient period through D15.
Document each opioid prescription provided to the patient and the number of and type of tablets provided to the patient for site records and reconciliation.

### 3. PROs: AAS, PROMIS 10 Global at Screening, T48, T96, D8, D15, and D29.

### 4. PBOMs: Sit to Stand Test, TUG at T48 (±4 h), D5/Discharge, D8, D15, and D29.

### Study Results:

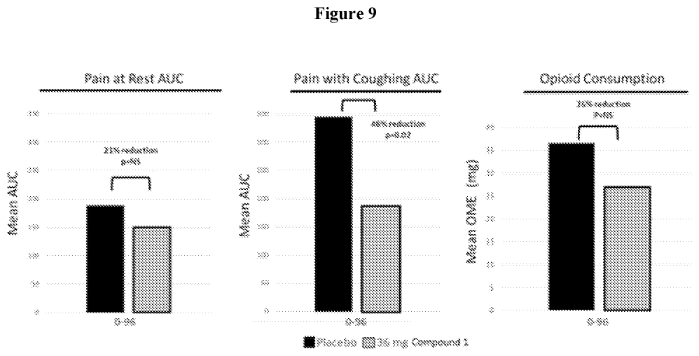

Compound 1 at a dose of 36 mg (0.3 mg/mL) demonstrated long lasting reductions in pain in patients undergoing surgery to repair a ventral hernia. At a dose of 36 mg (0.3 mg/mL), Compound 1 achieved a 21% reduction in pain at rest and a 46% reduction in pain with coughing compared to placebo (FIG. 9). In addition, patients treated with Compound 1 achieved a 26% reduction in opioid consumption compared to placebo (FIG. 9).

The examples and embodiments described herein are for illustrative purposes only and in some embodiments, various modifications or changes are to be included within the purview of the disclosure and scope of the appended claims.

What is claimed is:

1. A method of treating pain in a subject undergoing surgery comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.05 mg/mL to 0.5 mg/mL.

2. The method of claim 1 comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.1 mg/mL to 0.4 mg/mL.

3. The method of claim 1 or claim 2 comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.15 mg/mL to 0.35 mg/mL.

4. The method of any one of claims 1-3 comprising administering to the subject (E)-2-methoxy-4-((8-methylnon-6-enamido)methyl)phenyl 2-((methylamino)methyl)piperidine-1-carboxylate (Compound 1) in an aqueous pharmaceutical formulation at a concentration of 0.2 mg/mL to 0.3 mg/mL.

5. The method of any one of claims 1-4 wherein the pain is from orthopedic surgery.

6. The method of any one of claims 1-5 wherein the pain is pain from a bunionectomy.

7. The method of any one of claims 1-5 wherein the pain is pain from a unilateral total knee arthroplasty (TKA).

8. The method of any one of claims 1-3 wherein the pain is from a laparotomy.

9. The method of claim 8, wherein the pain from a laparotomy to repair a ventral hernia.

10. A method of treating pain in a subject undergoing a bunionectomy comprising administering to the subject 0.5 mg to 5.0 mg of (E)-2-methoxy-4-((8-methylnon-6-enamido)methyl)phenyl 2-((methylamino)methyl)piperidine-1-carboxylate (Compound 1).

11. A method of treating pain in a subject undergoing a bunionectomy comprising administering to the subject 0.5 mg to 5.0 mg of (E)-2-methoxy-4-((8-methylnon-6-enamido)methyl)phenyl 2-((methylamino)methyl)piperidine-1-carboxylate (Compound 1), wherein the pain is reduced by at least 15% compared to placebo.

12. The method of claim 11, wherein the pain is reduced by at least 20% compared to placebo.

13. The method of claim 11, wherein the pain is reduced by at least 25% compared to placebo.

14. The method of claim 11, wherein the pain is reduced by at least 30% compared to placebo.

15. A method of treating pain and reducing opioid use in a subject undergoing a bunionectomy, comprising administering to the subject 0.5 mg to 5.0 mg of (E)-2-methoxy-4-((8-methylnon-6-enamido)methyl)phenyl 2-((methylamino)methyl)piperidine-1-carboxylate (Compound 1), wherein opioid use is reduced by at least 20%.

16. The method of claim 15, wherein opioid use is reduced by at least 30%.

17. The method of claim 15, wherein opioid use is reduced by at least 40%.

18. The method of claim 15, wherein opioid use is reduced by at least 50%.

19. The method of any one of claims 10-18 comprising administering to the subject 2.0 mg to 5.0 mg of (E)-2-methoxy-4-((8-methylnon-6-enamido)methyl)phenyl 2-((methylamino)methyl)piperidine-1-carboxylate.

20. The method of any one of claims 10-19 comprising administering to the subject 3.0 mg to

5. 0 mg of (E)-2-methoxy-4-((8-methylnon-6-enamido)methyl)phenyl 2-((methylamino)methyl)piperidine-1-carboxylate.

21. The method of any one of claims 10-20 comprising administering to the subject 4.0 mg to 5.0 mg of (E)-2-methoxy-4-((8-methylnon-6-enamido)methyl)phenyl 2-((methylamino)methyl)piperidine-1-carboxylate.

22. A method of treating pain in a subject undergoing a unilateral total knee arthroplasty (TKA) comprising administering to the subject 25 mg to 65 mg of (E)-2-methoxy-4-((8-methylnon-6-enamido)methyl)phenyl 2-((methylamino)methyl)piperidine-1-carboxylate (Compound 1).

23. A method of treating pain in a subject undergoing a unilateral total knee arthroplasty (TKA) comprising administering to the subject 25 mg to 65 mg of (E)-2-methoxy-4-((8-methylnon-6-enamido)methyl)phenyl 2-((methylamino)methyl)piperidine-1-carboxylate (Compound 1), wherein the pain is reduced by at least 10% compared to placebo.

24. The method of claim 23, wherein the pain is reduced by at least 15% compared to placebo.

25. The method of claim 23, wherein the pain is reduced by at least 20% compared to placebo.

26. A method of treating pain and reducing opioid use in a subject undergoing a unilateral total knee arthroplasty (TKA) comprising administering to the subject 25 mg to 65 of (E)-2-methoxy-4-((8-methylnon-6-enamido)methyl)phenyl 2-((methylamino)methyl)piperidine-1-carboxylate (Compound 1), wherein opioid use is reduced by at least 10%.

27. The method of claim 26, wherein opioid use is reduced by at least 20%.

28. The method of claim 26, wherein opioid use is reduced by at least 30%.

29. The method of any one of claims 22-28 comprising administering to the subject 25 mg to 50 mg of (E)-2-methoxy-4-((8-methylnon-6-enamido)methyl)phenyl 2-((methyl amino)methyl)piperi dine-1-carboxylate.

30. The method of any one of claims 22-29 comprising administering to the subject 30 mg to 40 mg of (E)-2-methoxy-4-((8-methylnon-6-enamido)methyl)phenyl 2-((methyl amino)methyl)piperi dine-1-carboxylate.

31. A method of treating pain in a subject undergoing a laparotomy comprising administering to the subject 25 mg to 65 mg of (E)-2-methoxy-4-((8-methylnon-6-enamido)methyl)phenyl 2-((methylamino)methyl)piperidine-1-carboxylate (Compound 1).

32. A method of treating pain in a subject undergoing a laparotomy comprising administering to the subject 25 mg to 65 mg of (E)-2-methoxy-4-((8-methylnon-6-enamido)methyl)phenyl 2-((methylamino)methyl)piperidine-1-carboxylate (Compound 1), wherein the pain is reduced by at least 10% compared to placebo.

33. The method of claim 32, wherein the pain is reduced by at least 15% compared to placebo.

34. The method of claim 33, wherein the pain is reduced by at least 20% compared to placebo.

35. A method of treating pain and reducing opioid use in a subject undergoing a laparotomy comprising administering to the subject 25 mg to 65 of (E)-2-methoxy-4-((8-methylnon-6-enamido)methyl)phenyl 2-((methylamino)methyl)piperidine-1-carboxylate (Compound 1), wherein opioid use is reduced by at least 10%.

36. The method of claim 35, wherein opioid use is reduced by at least 20%.

37. The method of claim 36, wherein opioid use is reduced by at least 30%.

38. The method of any one of claims 31-37, wherein the subject is undergoing laparotomy to repair a ventral hernia.

39. The method of any one of claims 31-38 comprising administering to the subject 25 mg to 50 mg of (E)-2-methoxy-4-((8-methylnon-6-enamido)methyl)phenyl 2-((methylamino)methyl)piperidine-1-carboxylate.

40. The method of any one of claims 31-39 comprising administering to the subject 30 mg to 40 mg of (E)-2-methoxy-4-((8-methylnon-6-enamido)methyl)phenyl 2-((methylamino)methyl)piperidine-1-carboxylate.

41. The method of any one of claims 1-40, wherein (E)-2-methoxy-4-((8-methylnon-6-enamido)methyl)phenyl 2-((methylamino)methyl)piperidine-1-carboxylate (Compound 1) is administered by infiltration and instillation.

42. The method of claim 41, wherein 10% to 35% of (E)-2-methoxy-4-((8-methylnon-6-enamido)methyl)phenyl 2-((methylamino)methyl)piperidine-1-carboxylate (Compound 1) is administered by instillation and 65% to 90% of (E)-2-methoxy-4-((8-methylnon-6-enamido)methyl)phenyl 2-((methylamino)methyl)piperidine-1-carboxylate (Compound 1) is administered by infiltration.

43. The method of claim 42, wherein 15% to 30% of (E)-2-methoxy-4-((8-methylnon-6-enamido)methyl)phenyl 2-((methylamino)methyl)piperidine-1-carboxylate (Compound 1) is administered by instillation and 70% to 85% of (E)-2-methoxy-4-((8-methylnon-6-enamido)methyl)phenyl 2-((methylamino)methyl)piperidine-1-carboxylate (Compound 1) is administered by infiltration.
